(19) United States
(12) Patent Application Publication
(10) Pub. No.: US 2023/0393076 A1
(43) Pub. Date: Dec. 7, 2023

(54) AUTOMATED ONLINE MINERAL SLURRY AND PROCESS WATER PH ANALYZER, QUANTITATIVE VOLUMETRIC TITRATION ANALYZER, AND LIQUID HARDNESS ANALYZER

(71) Applicant: THE SASKATCHEWAN RESEARCH COUNCIL, Saskatoon (CA)

(72) Inventors: Ruijun SUN, Saskatoon (CA); WILLIAMS David, Saskatoon (CA); MCGILP Lesley, Saskatoon (CA)

(21) Appl. No.: 18/021,787

(22) PCT Filed: Aug. 18, 2021
(86) PCT No.: PCT/CA2021/051145

§ 371 Date: Feb. 16, 2023

Related U.S. Application Data
(60) Provisional application No. 63/067,545, filed on Aug. 19, 2020.

(51) Int. Cl. G01N 21/80; G01N 1/20; G01N 1/38; G01N 33/18

(52) U.S. Cl. CPC G01N 21/80; G01N 1/2035; G01N 1/38; G01N 33/1853; G01N 2001/2057; G01N 2001/4088

(57) ABSTRACT

Automated analyzers to measure or determine parameters in mineral slurries or process water, in particular to online and automated analyzers to measure pH, or to perform quantitative volumetric titrations relying on spectra absorbance of a liquid extracted from titrant and titrant mixture to determine the endpoint of titration, such as the measurement of liquid hardness in mineral slurries or process water. An automated pH analyzer may include a processor operable to manage the operations associated with the apparatus, an automated sampler coupled to the vessel or conduit and operable to extract a sample of a determined volume of the slurry or process water from the vessel or conduit, the automated sampler being under control of the processor, a water source under control of the processor and operable to deliver a known volume of water of a known pH into the sample, a mixing chamber that receives the known volume of water and the sample, an agitator operable to agitate the sample and the known volume of water in the mixing chamber to produce a diluted sample mixture, an automated filter operable to extract an aliquot of the diluted sample mixture from the mixing chamber and to filter the aliquot to produce a filtrate, a pH probe after the automated filter to measure the pH of filtrate, and a pH probe within the mixing chamber operable to measure a pH of the diluted sample mixture. The measurement is used to calculate the pH of the extracted sample, and to alter in near real time a process control of the a mineral processing operation related to the mineral slurry or process water.

### TECHNICAL FIELD

The technical field generally relates to online and automated analyzers to measure or determine parameters in mineral slurries or process water, in particular to online and automated analyzers to measure pH, or to perform quantitative volumetric titrations relying on spectra absorbance of a liquid extracted from titrant and titrant mixture to determine the endpoint of titration, such as the measurement of liquid hardness in mineral slurries or process water.

### BACKGROUND

### pH Measurement

Online pH measurement on slurries or process water containing non-aqueous liquids and/or abrasive solid particles face several challenges. Firstly, non-aqueous liquids such as hydrocarbons in the sample could destabilize readings, delay response, and generate incorrect pH results. This is because hydrocarbons in the slurry sample reduce liquid ion strength and conductivity, causing the pH probe to be less sensitive to [H⁺] and [OH⁻ ] ion concentration change; hydrocarbons could also dehydrate the electrode membrane and increase liquid junction potentials. In some applications, oil or bitumen in slurry or process water could coat and/or disable the pH probe in a very short time. Secondly, abrasive solid particles in a flowing slurry could erode the electrode membrane and disable the pH probe, shorten the lifespan of pH probe installed in the flowing slurry. These challenges make the direct pH measurement in some slurry or process water applications very difficult.

Attempts have been made to improve the design and materials used for pH probes so that they can be used in partial non-aqueous applications. While some of these pH probes have limited successes, they do not resolve the issues of bitumen/oil coating and solid particle attrition, and remain unsuitable for applications such as oil sands operations or other operations in which the mineral slurries contain hydrocarbons and/or abrasive solid particles. Therefore, there is a need for an improved online pH metering apparatus to measure the pH of slurries or process water containing non-aqueous liquids and/or abrasive solid particles. Accordingly, there is a need for an automated online mineral slurry and process water pH analyzer that measures pH indirectly for slurries containing non-aqueous liquids and abrasive solids particles and that provides results as close to real time as possible. Such pH analyzer would be advantageous in order to achieve better process control and save operating cost, as well as other benefits apparent to persons skilled in the art.

### Quantitative Volumetric Titrations

Some quantitative volumetric titrations relying on accurate determination of titration endpoint and correlating the endpoint to titrant volume which can be used as a process control parameter. Such quantitative volumetric titration play an important role in mineral processing and process control, which typically involve manual procedures and/or calculations, require skilled personnel to perform, and are time consuming to yield results, rendering them unsuitable for close to real-time information processing and process control.

Accordingly, there is a need for an automated online mineral slurry and process water quantitative volumetric titration analyzer that automatically performs a titration and determines an endpoint of the titrant volume based on changes in liquid spectra absorbance and correlates the endpoint to one or more parameters of the mineral slurry or process water. Such automated online mineral slurry and process water quantitative volumetric titration analyzer would be advantageous in order to achieve better process control and save operating cost, as well as other benefits apparent to persons skilled in the art.

### Water Hardness

Water hardness metal ions such as Calcium and Magnesium could present challenges in water supply to water heating equipment, such as for example boilers and heat exchangers, causing equipment and pipe scaling and clogging. If not detected and treated, water hardness could result in the reduction of process efficiency and/or heat transfer. For example, in the Canadian oil sands industry, the Steam Assisted Gravity Drainage (SAGD), Enhanced Oil Recovery (EOR) and Cyclic Steam Stimulation (CSS) processes use large quantities of steam for oil extraction operations, water hardness monitoring becomes even more important to prevent corrosion and scale build-up, not only to ensure efficient and cost-effective operation, but also to improve environmental performance of the oil sands operation and to reduce greenhouse emission.

To reduce impact of water hardness, chemicals such as lime are used to soften the water; however, the dose of water softeners need to be carefully controlled to minimize the cost of water softening, reduce its effect on process water recycle and reuse, and to minimize its impact on the environment since, inevitably, the process water and added chemicals will be discharged into the environment. Therefore, there is a need to monitor process water hardness and control the dose of water softeners.

There are two major categories of test methods that are presently used in the steam-assisted oil sands processes to determine the water hardness. One category utilizes instrumentation laboratory analysis such as Inductively Coupled Plasma (ICP) Spectroscopy. The water hardness is determined from Calcium and Magnesium ion concentrations measured by ICP. However, ICP requires considerable resources and qualified personnel to operate, it has high requirements for the sample as any impurities in the sample could interfere with the results, and it takes hours for sample preparation and results generation. Therefore, ICP is not robust enough to be easily adapted as an online instrument and may not be suitable for harsh environment at some of the application sites.

The conventional category of measuring hardness in water is by complexometric titration, a form of volumetric titration analysis mentioned above, in which the endpoint of the titration is indicated by formation of a coloured complex, as outlined by ASTM D 1126-17 “Standard Test Method for Hardness in Water” and other publications. The water sample is chemically conditioned by adjusting pH to 7-10 by adding NH₄OH or HCl solutions and buffer solution, followed by adding a dose of water hardness indicator such as Eriochrome Black T (EBT). The EBT solution has a blue colour, or pink colour if the water used to prepare EBT solution contains trace amount of Calcium and/or Magnesium ions. After EBT molecules are complexed or bound with Calcium and/or Magnesium ions, the liquid sample changes colour from blue to pink/red. When a colourless chelating agent such as Ethylenediamine Tetraacetic Acid (EDTA) solution is titrated into the sample, it un-complexes the bonding between Calcium/Magnesium and EBT, because EDTA binds more strongly with Calcium and Magnesium ions, thus releasing the EBT molecules into the sample solution. When the un-complexing process completes at the titration endpoint, the sample liquid changes colour from pink/red to blue again. The Calcium and/or Magnesium ion concentrations can be determined from the concentration and cumulative volume of EDTA solution titrated to reach the endpoint and reported either as total hardness (Calcium and Magnesium combined) or the hardness portion contributed by Calcium or Magnesium individually.

The complexometric titration is conducted by laboratory procedures that require manual sample preparation, transferring and titration, and the endpoint is identified visually. It can be performed manually by off-site laboratory. The human detection of the endpoint could be subjective and erroneous as the procedures could be affected by many parameters and measuring conditions. For water samples containing fine oil droplets and/or residual particles that could interfere with the colorimetric analysis, more sample processing and preparation procedures are required to remove oil droplets and particles. Therefore, the current ASTM D 1126-17 method for measuring the hardness in water is not an automated and online method and not for oil sands process water without sample pre-treatment.

Accordingly, there is a need for an automated online mineral slurry and process water hardness analyzer that automatically measure hardness, Calcium and Magnesium ion concentrations in liquid, and that provides results as close to real time as possible. Such automated online water hardness analyzer would be advantageous in order to achieve better process control and save operating cost, as well as other benefits apparent to persons skilled in the art.

### SUMMARY

Accordingly, in some aspects, the present invention provides an automated and online mineral slurry and process water pH analyzer in which a fixed volume of slurry or process water sample, for which the pH is to be determined, is automatically withdrawn from a process. The sample is carried by a controlled volume of a dilution water with a known pH to a mixing chamber where the diluted sample is thoroughly mixed, and the pH of the diluted sample mixture is measured. The measured pH of the diluted sample mixture is used to calculate the pH of the withdrawn process sample. This is because the hydroxide [OH⁻] ion concentrations in the diluted sample mixture can be determined by measuring the liquid pH. This [OH⁻] ion concentration in the mixture, along with the known volumes of process water and dilution water, and the known pH and [OH⁻] of dilution water, can be used to determine the hydroxide [OH⁻] ion concentration in the process sample that is then convert it to a pH value. In some aspects, the process sample is withdrawn and transferred directly into the mixing chamber without dilution, followed by automatically extracting a filtrate and measure its pH which is the actual process pH. The present invention provides close to real-time online measurement of pH in a process when direct measurement of process pH is not feasible due to hydrocarbon coating on the pH probe and/or attrition from the solid particles contained in the slurry of the process flow.

In some aspects, the present invention provides an automated online mineral slurry and process water volumetric titration analyzer that automatically performs titrations on a sample of mineral slurry or process water and determines an endpoint of the titration based on changes in liquid spectra absorbance, and correlates the endpoint to one or more parameters of the mineral slurry or process water. A controlled volume of slurry or process water sample is automatically withdrawn from the process and mixed with water at a controlled volume. The diluted mixture sample is then conditioned with chemicals and/or chemical indicator, and/or has its temperature regulated, as required, followed by injecting in increments a controlled volume of analytical reagent or titrant solution. After each titrant solution injection, a filtrate is extracted from the mixture. The filtrate spectra absorbance is measured by a spectrophotometer and correlated to the cumulative volume of the titrant solution and/or a parameter of the mineral slurry or process water. The online systems can be operated automatically and continuously to achieve better process control through rapid response to process condition change, save water and operation cost, and minimize process failure.

In some embodiments, the quantitative volumetric titration analyzer is an automated and online water hardness analyzer that determines water hardness in liquids in mineral slurries or process water. A controlled volume of slurry or process water sample is automatically withdrawn from the process and mixed with water at controlled volume. The diluted mixture is then conditioned with chemicals and indicators, such as Eriochrome Black T (EBT), followed by injecting in increments a controlled volume of titrant such as Ethylenediamine Tetraacetic Acid (EDTA) solution. After each EDTA injection, a filtrate is extracted from the mixture. The filtrate spectra absorbance is measured by a spectrophotometer and correlated to the cumulative EDTA volume and generate the liquid hardness value. The online systems can be operated automatically and continuously to achieve better process control through rapid response to process condition change, save water and operation cost, and minimize process failure.

In the case of the online water hardness analyzer, a fixed volume of slurry or process water sample, for which the water hardness is to be determined, is automatically withdrawn from a process. The sample is carried by a controlled volume of a dilution water to a mixing chamber where the diluted sample is thoroughly mixed. In case the process sample is super-hot, sample temperature can be reduced and adjusted by the dilution water which is regulated either in the supply container and/or via a thermal jacket installed on the transfer line as well as in the mixing chamber. The diluted sample mixture is further conditioned by injecting chemical solutions until it reaches a target pH, followed by injecting a controlled dose and volume of liquid hardness indicator such as Eriochrome Black T (EBT). A controlled dose and volume of liquid chelating agent such as Ethylenediamine Tetraacetic Acid (EDTA) is then injected in increments into the conditioned mixture. After each EDTA injection and dispersion, a small aliquot is extracted, filtered and the filtrate is measured by a spectrophotometer. The spectra absorbance of the filtrate at a given wavelength can be used to determine the critical EDTA volume required to reach the titration endpoint when spectra absorbance vs. cumulative EDTA volume curve shows a transition and/or when the spectra absorbance reaches a critical value. The critical EDTA volume, sample volume and the EDTA dosage are used to determine the total hardness, Calcium and Magnesium hardness in the liquid according to ASTM D1126-17 for process control purpose.

The water hardness analyzer can be installed online on a live slurry pipeline, mixing vessel, water supply tank and/or pipeline, and can analyze slurry or liquid sample automatically and continuously. The analyzer system comprises of an automated sampler, a mixing chamber equipped with a mixer, two pH probes (A and B), containers to supply dilution water, conditioning chemicals, EBT and EDTA solutions, an automated filtration device, a spectrophotometer with a flowcell, a data transmitter, and a processor to perform computations on the measured spectral absorbance data. The water hardness analyzer of the present invention provides close to real-time measurement of total hardness, Calcium and Magnesium ion concentration, this enables online monitoring of liquid hardness in slurry or process water.

An online slurry and process water pH analyzer and/or the liquid hardness analyzer in accordance with embodiments of the present invention automatically take a controlled volume of slurry or process water from the process pipeline or container, dilute the sample by dilution water with a controlled volume and pH, mix the sample and dilution water in a mixing chamber and analyze the pH of diluted mixture. The pH of the diluted mixture is measured and correlated to determine the pH of slurry or process water in the process where direct measurement of process pH is not feasible due to coating on the pH probe by hydrocarbons and/or attritions by abrasive sand particles contained in the process flow.

In the case of the liquid hardness analyzer, it can determine the liquid hardness in the process by measuring the spectra absorbance of a filtrate extracted from the process sample after treated with chemicals, indicator such as EBT and titrant such as EDTA.

In each of the pH analyzer and the quantitative volumetric titration analyzer, an automated sampler such as an Isolok™ sampler, is configured to withdraw a fixed volume of slurry sample from a live slurry pipeline or mixing vessel, or process water supply line or tank. A water container is configured to receive and hold dilution water used to dilute the slurry sample and to flush out the apparatus after each sample analysis. A controlled volume and pH of dilution water is dispensed into the automated sampler to flush out the sample and carry the diluted slurry or process water sample into a mixing chamber that is provided with an agitator or mixer and a pH probe A. The diluted slurry sample is mixed in the mixing chamber by the mixer.

In the case of the pH analyzer, the pH of diluted sample is measured by the pH probe A and the value is used to determine the pH of process sample based on the known process sample volume, dilution water volume and pH, and liquid content of the sample from its density measured by a densitometer installed near the sampler. In some aspects, the process sample is withdrawn and transferred directly into the mixing chamber without dilution, followed by automatically extracting a filtrate and utilizing pH probe B to measure the filtrate pH which is the actual process pH.

In the case of the water hardness analyzer, several containers are configured to receive and hold different chemical solutions and to dispense said chemical solutions in controlled volumes into the mixing chamber. Controlled volumes of chemical solutions are dispensed in increments into the diluted sample in the mixing chamber until the pH of the diluted mixture as measured by the pH probe A reaches target values. The mixing continue until a target duration is reached based on pre-calibrations. A hardness indicator (such as EBT) solution container is configured to hold the EBT solution and to dispense the EBT solution into the slurry mixture in the mixing chamber in controlled volume. While mixing, a controlled volume of EBT solution is dispensed into the diluted mixture. An EDTA solution container is configured to hold EDTA solution. While mixing, a controlled volume of EDTA solution is dispensed in increments into the diluted mixture. After each EDTA solution injection and mixing, an aliquot of analyte is removed from the sample mixture through an automated filter and the filtrate is transferred into an optical flowcell of a spectrophotometer. The filtrate is measured by the spectrophotometer and the spectra absorbance data is transmitted to a computer for storage and computational analysis. The steps of EDTA solution injection to the sample mixture, filtrate removing and measuring by the spectrophotometer is repeated to obtain a series of spectra absorbance data as well as EDTA cumulative volume for processing by the computer. The EDTA solution injection is stopped after the filtrate spectra absorbance passes a target value (endpoint), or enough spectra absorbance data is obtained to enable useful correlation. The endpoint and/or the spectra absorbance obtained before and after reaching the endpoint, along with other parameters determined (e.g., cumulative volume of EDTA solution injected, density and temperature of the slurry, etc.) by other instruments installed in the system, can be used to correlate and determine the liquid total hardness, Calcium hardness, Magnesium hardness and hardness as Calcium Carbonate, as well as other parameters.

In the case of the pH analyzer or the liquid hardness analyzer, after the sample pH or liquid hardness value is determined, another controlled volume of water, with or without a dose of solvent and/or detergent if necessary, is flushed into the automated sampler and through the mixing chamber to wash out the spent sample mixture through a drainage port provided in the mixing chamber. The flushing water also washes and cleans the automated sampler, the mixer impeller, pH probe, the automated filter and the mixing chamber interior while the mixer is actuated. After the water flush, the online mineral slurry and process water pH and hardness analyzer is ready to analyze another sample.

In some aspects the present invention provides an automated pH analyzer for determining the pH in a mineral slurry or process water in a vessel or passing through a conduit, the apparatus comprising: a processor operable to manage the operations associated with the apparatus; an automated sampler coupled to the vessel or conduit and operable to extract a sample of a determined volume of the slurry or process water from the vessel or conduit, the automated sampler being under control of the processor; a water source under control of the processor and operable to deliver a known volume of water of a known pH into the sample; a mixing chamber that receives the known volume of water and the sample; an agitator operable to agitate the sample and the known volume of water in the mixing chamber to produce a diluted sample mixture; an automated filter operable to extract an aliquot of the diluted sample mixture from the mixing chamber and to filter the aliquot to produce a filtrate; a pH probe after the automated filter to measure the pH of filtrate; and a pH probe within the mixing chamber operable to measure a pH of the diluted sample mixture, wherein the measurement is used in any one or more of following: to calculate the pH of the extracted sample, and to alter in near real time a process control of the a mineral processing operation related to the mineral slurry or process water.

In some embodiments, the apparatus may be online such that the sample is withdrawn from an online active process.

In some embodiments, the processor may be operable to instruct the automated sampler to extract the sample from the vessel or conduit.

In some embodiments, the water source may deliver the known volume and known pH of water to the automated sampler after the sample has been extracted to flush the sample out of the automated sampler and into the mixing chamber.

In some embodiments, the processor may be operable to instruct the water source to deliver the known volume of water to the automated sampler.

In some embodiments, the water source may cooperate with the automated sampler to deliver the volume of water into the extracted sample to flush it out of the automated sampler to clean the automated sampler thereby ready it for obtaining a subsequent sample of slurry or process water.

In some embodiments, the agitator may be controlled by the processor.

In some embodiments, the processor may be operable to activate the agitator to mix the sample mixture after the sample mixture is received in the mixing chamber.

In some embodiments, the processor may be operable to receive the pH measurement of the diluted sample mixture from the pH probe within the mixing chamber after a period of agitation of the diluted sample mixture.

In some embodiments, the water source may be operable to flush water through one or both of the automated sampler and the mixing chamber, after the pH measurement of the diluted sample mixture, to clean one or both of the automated sampler and the mixing chamber in preparation for processing a subsequent sample.

In some embodiments, the processor may be operable to activate the agitator while the water source is operable to flush water through the mixing chamber.

In some embodiments, the automated filter may further comprise a second automated sampler coupled to the mixing chamber and operable to extract the aliquot from the mixing chamber after mixing the process sample with dilution water; and a filter element downstream of the automated sampler, wherein to produce a filtrate and the pH of filtrate is measured by the pH probe installed after the automated filter.

In some embodiments, the processor may be operable to calculate the pH of the sample using the known volume of the sample, the known volume of the water delivered into the sample, the known pH of the volume of water delivered into the sample, the measured pH of the diluted sample mixture, and the measured pH of the filtrate.

In some aspects the present invention provides a method of determining a pH in a mineral slurry or process water in a vessel or passing through a conduit, the method comprising: (a) coupling an automated sampler with the vessel or conduit such that the automated sampler is operable to extract a sample of a known volume of the slurry or process water from the vessel or conduit; (b) providing instructions from a processor to the automated sampler to extract the sample; (c) flushing the sample from the automated sampler into a mixing chamber with a known volume of water having a known pH from a water source under control of the processor; (d) mixing the sample and the volume of water with an agitator in the mixing chamber under control of the processor to produce a diluted sample mixture; (e) measuring a pH of the diluted sample mixture with a pH probe in the mixing chamber under control of the processor; (f) extract an aliquot of the sample mixture, filter through an automated filter and measure the pH of filtrate by a pH probe after the automated filter; and (g) analyzing the pH measurement of the diluted sample mixture and filtrate with the processor to determine a pH of the extracted process sample.

In some embodiments, the method may further comprise flushing water from the water source under control of the processor through the automated sampler and mixing chamber after step (f) to expel remnants of the diluted sample mixture therefrom in preparation for processing a subsequent sample.

In some aspects the present invention provides an automated quantitative volumetric titration analyzer for performing automated quantitative volumetric titrations of a mineral slurry or process water in a vessel or passing through a conduit, the apparatus comprising: a processor operable to manage the operations associated with the apparatus; an automated sampler coupled to the vessel or conduit and operable to extract a sample of a determined volume of the slurry or process water from the vessel or conduit, the automated sampler being under control of the processor; a water source under control of the processor and operable to deliver a known volume of water into the sample; a titrant solution source under control of the processor and operable to deliver a known volume of titrant solution to the sample; a mixing chamber that receives the sample, the water, and the titrant solution; an agitator operable to agitate the sample, the water, and the titrant solution in the mixing chamber to produce a diluted sample mixture; an automated filter operable to extract an aliquot of the diluted sample mixture from the mixing chamber and to filter the aliquot to produce a filtrate; and a spectrophotometer having an optical flowcell that receives the filtrate from the automated filter and operable to measure a spectra absorbance of the filtrate in the optical flowcell using at least one wavelength to obtain spectra absorbance data of the filtrate.

In some embodiments, the apparatus may be online such that the sample is withdrawn from an online active process.

In some embodiments, the apparatus may further comprise a source of chemicals under control of the processor and operable to deliver chemicals into the mixing chamber for chemically conditioning the sample mixture.

In some embodiments, the apparatus may further comprise a pH probe within the mixing chamber operable to measure a pH of the diluted sample mixture, wherein the processor is operable to control the delivery of chemicals to the sample mixture based on the pH measurement.

In some embodiments, the apparatus may further comprise: a recirculating chiller coupled to the mixing chamber operable to heat or cool the sample mixture; a temperature probe in the mixing chamber operable to measure a temperature of the sample mixture; and wherein the processor is operable to receive the temperature measurement from the temperature probe and to activate the recirculating chiller based on the temperature measurement to achieve a desired temperature of the sample mixture.

In some embodiments, the processor may be operable to instruct the automated sampler to extract the sample from the vessel or conduit.

In some embodiments, the water source may deliver the known volume of water to the automated sampler after the sample has been extracted to flush the sample out of the automated sampler and into the mixing chamber.

In some embodiments, the processor may be operable to instruct the water source to deliver the known volume of water to the automated sampler.

In some embodiments, the water source may cooperate with the automated sampler to deliver the volume of water into the extracted sample to flush it out of the automated sampler to clean the automated sampler thereby ready it for obtaining a subsequent sample of slurry or process water.

In some embodiments, the agitator may be controlled by the processor.

In some embodiments, the processor may be operable to activate the agitator to mix the sample mixture after the sample mixture is received in the mixing chamber.

In some embodiments, the water source may be operable under control of the processor to flush water through one or both of the automated sampler and the mixing chamber to clean one or both of the automated sampler and the mixing chamber in preparation for processing a subsequent sample.

In some embodiments, the processor may be operable to activate the agitator while the water source is operable to flush water through the mixing chamber.

In some embodiments, the automated filter may comprise: a second automated sampler coupled to the mixing chamber and operable to extract the aliquot from the mixing chamber after each delivery of the titrant solution; and a filter element downstream of the second automated sampler, wherein the second automated sampler pumps the aliquot through the filter element and the filtrate to the optical flowcell for obtaining spectra absorbance measurements of each filtrate. In some embodiments, the automated filter may include a pressure sensor that senses pressure of the aliquot upstream of the filter element; and a mechanism operable to replace the filter element with a fresh filter element as a result of a signal from the pressure sensor that the pressure of the aliquot has increased beyond a threshold pressure.

In some embodiments, the processor may be operable to determine a titration endpoint from the spectra absorbance data.

In some embodiments, the processor may be operable to control a processing of the mineral slurry or process water or to control in near real time a processing operation related to the mineral slurry or process water, based on the spectra absorbance data.

In some embodiments, if the processor determines the titration endpoint has not been reached, the processor may be further operable: to instruct the titrant solution source to deliver an additional known volume of titrant solution to the dilute sample mixture; thereafter to instruct the automated filter to obtain a subsequent aliquot of the diluted sample mixture and filter same to produce a subsequent filtrate; and thereafter to instruct the spectrophotometer to measure a spectra absorbance of the subsequent filtrate to obtain a subsequent spectra absorbance data; and thereafter determine if the titration endpoint has been reached from the subsequent spectra absorbance data.

In some embodiments, if the processor determines the titration endpoint has been reached and/or enough titration data has been obtained, the processor may be further operable to instruct the water source to flush water through one or both of the automated sampler and the mixing chamber to clean one or both of the automated sampler and the mixing chamber in preparation for processing a subsequent sample of mineral slurry or process water.

In some aspects the present invention provides a method of automatically performing a quantitative volumetric titration on a mineral slurry or process water in a vessel or passing through a conduit, the method comprising the steps of: (a) coupling an automated sampler with the vessel or conduit such that the automated sampler is operable to extract a sample of a known volume of the slurry or process water from the vessel or conduit; (b) providing instructions from the processor to the automated sampler to extract the sample; (c) flushing the sample from the automated sampler into a mixing chamber with a known volume of water from a water source under control of the processor; (d) mixing the sample and water in the mixing chamber to produce a diluted sample mixture; (e) adding a known volume of chemical and indicator solutions into the diluted sample mixture from chemical and indicator solution sources under control of the processor; (f) adding a known volume of a titrant solution into the diluted sample mixture from a titrant solution source under control of the processor; (g) filtering an aliquot of the diluted sample mixture through filter media of an automated filter and directing a filtrate of the aliquot into an optical flowcell of a spectrophotometer; (h) measuring spectra absorbance of the filtrate under control of the processor to obtain spectra absorbance data of the filtrate, and storing the spectra absorbance data in memory; (i) repeating steps (f) to (h) until a target spectra absorbance value or a plurality of target spectra absorbance values is reached to obtain a spectra absorbance data set; (j) flushing water through the automated sampler and mixing chamber to expel remnants of the slurry sample and process solutions therefrom in preparation for processing a subsequent sample; and (k) analyzing the spectra absorbance data set and using a result of the analysis in controlling processing of the mineral slurry or process water or controlling other aspects of a mineral processing operation related to the mineral slurry or process water.

In some embodiments, the method may further comprise a step of homogenizing the sample mixture before and after adding titrant solution to disperse particles in the sample mixture.

In some embodiments, the step of homogenizing the sample mixture may take place in the mixing chamber.

In some embodiments, the method may further comprise a step of measuring a density of the slurry sample in the vessel or conduit near the analyzer.

In some embodiments, the method may further comprise regulating a temperature of the sample mixture in the mixing chamber under control from the processor.

In some embodiments, the step of regulating a temperature of the diluted sample mixture may comprise establishing a flow of hot fluid or cold fluid through a fluid jacket provided around at least a portion of the mixing chamber.

In some embodiments, the method may further comprise repeating steps (b) to (j) to obtain a data set on a desired number of samples.

In some aspects the present invention provides an automated liquid hardness analyzer for determining the hardness in a mineral slurry or process water in a vessel or passing through a conduit, the apparatus comprising: a processor operable to manage the operations associated with the apparatus; an automated sampler coupled to the vessel or conduit and operable to extract a sample of a determined volume of the slurry or process water from the vessel or conduit, the automated sampler being under control of the processor; a water source under control of the processor and operable to deliver a known volume of water into the sample; an Eriochrome Black T (EBT) solution source under control of the processor and operable to deliver a known volume of EBT solution to the sample; an Ethylenediamine Tetraacetic Acid (EDTA) solution source under control of the processor and operable to deliver a known volume of EDTA solution to the sample; a mixing chamber that receives the sample, the water, the EBT solution and the EDTA solution; an agitator operable to agitate the sample, the water, the EBT solution and the EDTA solution in the mixing chamber to produce a diluted sample mixture; an automated filter operable to extract an aliquot of the diluted sample mixture from the mixing chamber and to filter the aliquot to produce a filtrate; a spectrophotometer having an optical flowcell that receives the filtrate from the automated filter and operable to measure a spectra absorbance of the filtrate in the optical flowcell using at least one wavelength to obtain spectra absorbance data of the filtrate; and wherein the processor if operable to determine the EDTA titration endpoint from the spectra absorbance data and to correlate the EDTA titration endpoint and the cumulative EDTA solution volume to a liquid hardness value of the extracted sample.

In some embodiments, the apparatus may be online such that the sample is withdrawn from an online active process.

In some embodiments, the apparatus may further comprise a source of chemicals under control of the processor and operable to deliver chemicals into the mixing chamber for chemically conditioning the sample mixture.

In some embodiments, the apparatus may further comprise a pH probe within the mixing chamber operable to measure a pH of the diluted sample mixture, wherein the processor is operable to control the delivery of chemicals to the sample mixture based on the pH measurement.

In some embodiments, the apparatus may further comprise: a recirculating chiller coupled to the mixing chamber operable to heat or cool the sample mixture; a temperature probe in the mixing chamber operable to measure a temperature of the sample mixture; and wherein the processor is operable to receive the temperature measurement from the temperature probe and to activate the recirculating chiller based on the temperature measurement to achieve a desired temperature of the sample mixture.

In some embodiments, the processor may be operable to instruct the automated sampler to extract the sample from the vessel or conduit.

In some embodiments, the water source may deliver the known volume of water to the automated sampler after the sample has been extracted to flush the sample out of the automated sampler and into the mixing chamber.

In some embodiments, the processor may be operable to instruct the water source to deliver the known volume of water to the automated sampler.

In some embodiments, the water source may cooperate with the automated sampler to deliver the volume of water into the extracted sample to flush it out of the automated sampler to clean the automated sampler thereby ready it for obtaining a subsequent sample of slurry or process water.

In some embodiments, the agitator may be controlled by the processor.

In some embodiments, the processor may be operable to activate the agitator to mix the sample mixture after the sample mixture is received in the mixing chamber.

In some embodiments, the water source may be operable under control of the processor to flush water through one or both of the automated sampler and the mixing chamber to clean one or both of the automated sampler and the mixing chamber in preparation for processing a subsequent sample.

In some embodiments, the processor may be operable to activate the agitator while the water source is operable to flush water through the mixing chamber.

In some embodiments, the automated filter may comprise: a second automated sampler coupled to the mixing chamber and operable to extract the aliquot from the mixing chamber after each delivery of the EDTA solution; and a filter element downstream of the second automated sampler, wherein the second automated sampler pumps the aliquot through the filter element and the filtrate to the optical flowcell for obtaining spectra absorbance measurements of each filtrate. In some embodiments, the automated filter may include a pressure sensor that senses pressure of the aliquot upstream of the filter element; and a mechanism operable to replace the filter element with a fresh filter element as a result of a signal from the pressure sensor that the pressure of the aliquot has increased beyond a threshold pressure.

In some embodiments, the processor may be operable to control a processing of the mineral slurry or process water in near real time based on the determined hardness value.

In some embodiments, if the processor determines the EDTA titration endpoint has not been reached, the processor may be further operable: to instruct the EDTA solution source to deliver an additional known volume of EDTA solution to the dilute sample mixture; thereafter to instruct the automated filter to obtain a subsequent aliquot of the diluted sample mixture and filter same to produce a subsequent filtrate; and thereafter to instruct the spectrophotometer to measure a spectra absorbance of the subsequent filtrate to obtain a subsequent spectra absorbance data; and thereafter determine if the titration endpoint has been reached from the subsequent spectra absorbance data.

In some embodiments, if the processor determines the EDTA titration endpoint has been reached, the processor may be further operable to instruct the water source to flush water through one or both of the automated sampler and the mixing chamber to clean one or both of the automated sampler and the mixing chamber in preparation for processing a subsequent sample of mineral slurry or process water.

In some aspects the present invention provides a method of automatically determining a liquid hardness value of a mineral slurry or process water in a vessel or passing through a conduit, the method comprising the steps of: (a) coupling an automated sampler with the vessel or conduit such that the automated sampler is operable to extract a sample of a known volume of the slurry or process water from the vessel or conduit; (b) providing instructions from the processor to the automated sampler to extract the sample; (c) flushing the sample from the automated sampler into a mixing chamber with a known volume of water from a water source under control of the processor; (d) mixing the sample and water in the mixing chamber to produce a diluted sample mixture; (e) adding known volume of chemical solutions into the diluted sample mixture from chemical solution source under control of the processor; (f) adding a known volume of Eriochrome Black T (EBT) solution into the diluted sample mixture from an EBT solution source under control of the processor; (g) adding a known volume of Ethylenediamine Tetraacetic Acid (EDTA) solution into the diluted sample mixture from an EDTA solution source under control of the processor; (h) filtering an aliquot of the diluted sample mixture through filter media of an automated filter and directing a filtrate of the aliquot into an optical flowcell of a spectrophotometer; (i) measuring spectra absorbance of the filtrate under control of the processor to obtain spectra absorbance data of the filtrate, and storing the spectra absorbance data in memory; (j) repeating steps (g) to (i) until a target spectra absorbance value or a plurality of target spectra absorbance values is reached to obtain a spectra absorbance data set; (k) flushing water through the automated sampler and mixing chamber to expel remnants of the sample and process solutions therefrom in preparation for processing a subsequent sample; and (l) analyzing the spectra absorbance data set and using a result of the analysis in determining a liquid hardness value for the extracted sample.

In some embodiments, the method may further comprise a step of homogenizing the sample mixture before and after adding titrant solution to disperse particles in the sample mixture.

In some embodiments, the step of homogenizing the sample mixture may take place in the mixing chamber.

In some embodiments, the method may further comprise a step of measuring a density of the slurry sample in the vessel or conduit near the analyzer.

In some embodiments, the method may further comprise regulating a temperature of the sample mixture in the mixing chamber under control from the processor.

In some embodiments, the step of regulating a temperature of the diluted sample mixture may comprise establishing a flow of hot fluid or cold fluid through a fluid jacket provided around at least a portion of the mixing chamber.

In some embodiments, the method may further comprise repeating steps (b) to (j) to obtain a data set on a desired number of samples.

In some embodiments, the automated filter is configured to connect to an air compressor; the aliquot of analyte is removed from the mixing chamber through the automated filter by air pressure;

In some embodiments, the filtrate is driven by the pressure generate from the automated filter and flow through the flowcell; in some embodiments, the filtrate is pumped through the flowcell by a peristaltic pump; in some embodiments, an additional mechanism automatically cleans the optical flowcell by injecting cleaning fluids periodically.

In some embodiments, Eriochrome Black T (EBT) is used as the water hardness titration indicator; in some embodiments, other indicators such as Patton-Reeder or other indicator is used as liquid hardness indicators.

In some embodiments, the spectra absorbance of filtrate is measured by the spectrophotometer and the data is transmitted to a computer; in some embodiments, the spectra absorbance of filtrate and cumulative volume of EDTA solutions injected, along with data of slurry sample volume, slurry density and temperature (both measured by other instruments commonly available in the system), are correlated to calculate the total hardness in liquid, Calcium and Magnesium hardness, Calcium and Magnesium ion concentrations, etc.

In some embodiments, the filtrate spectra absorbance reaches and passes a target value set by pre-calibrations, indicating an endpoint; in some embodiments, the filtrate spectra absorbances are part of the measurement which can be used for correlation and process control, the endpoint is not required.

In some embodiments, the liquid total hardness, Calcium and Magnesium hardness, Calcium and Magnesium ion concentrations can be used as input variables for the feedback or feed forward control systems to assist the controlling of slurry or process water parameters such as pressure, flowrate, temperature, blending ratio, chemicals and water softener dosages, pumping and mixing power, etc.

In some embodiments, the filtrate spectra absorbance measured by the spectrophotometer provide control signals for the number of increments and volume of EDTA solutions injection; in some embodiments, the filtrate spectra absorbance provides control signals to adjust the number of increments and volume of chemical solutions injection.

In some embodiments, the filtrate spectra absorbance provides control signals for the timing of slurry or process water sample and the volume of dilution water; in some embodiments, filtrate spectra absorbance provides control signals to adjust the mixing duration and power intensity.

The foregoing summary is illustrative only and is not intended to be in any way limiting. Other aspects and features of the present invention will become apparent to those of ordinary skill in the art upon review of the following description of embodiments of the invention in conjunction with the accompanying figures and claims.

### BRIEF DESCRIPTION OF THE DRAWINGS

In drawings which illustrate by way of example only embodiments of the invention:

FIG. 1 is a schematic illustration of an embodiment of an automated online mineral slurry and process water pH analyzer shown installed on a live slurry or process water pipeline;

FIG. 2 is a cross section of the pipeline and system in FIG. 1 showing an automated sampler taking a controlled volume of slurry or process water sample from a live pipeline (or other vessel) at controlled time intervals;

FIG. 3 is a cross section of the pipeline and system in FIG. 1 showing a controlled volume of dilution water with controlled pH value being injected into the automated sampler to dilute the slurry or process water sample and flush it into the mixing chamber;

FIG. 4 is a cross section of the pipeline and system in FIG. 1 showing the process sample is withdrawn and transferred directly into the mixing chamber with or without dilution, followed by automatically extracting a filtrate and measure the filtrate pH by pH probe B.

FIG. 5 is a process diagram of the automated and online mineral slurry and process water pH analyzer operation;

FIG. 6 is a schematic illustration of an embodiment of an automated online mineral slurry and process water hardness analyzer shown installed on a live slurry or process water pipeline;

FIG. 7 is a cross section of the pipeline and system in FIG. 6 showing an automated sampler taking a controlled volume of slurry or process water sample from a live pipeline (or other vessel) at controlled time intervals;

FIG. 8 is a cross section of the pipeline and system in FIG. 6 showing a controlled volume of dilution water being injected into the automated sampler to dilute the slurry or process water sample and flush it into the mixing chamber;

FIG. 9 is a schematic illustration of the chemicals and chemical indicator containers, and mixing chamber showing a controlled volume of chemicals solution injected in increments to the diluted slurry sample while mixing, the chemicals injection continues until a target pH value is reached as measured by pH probe A;

FIG. 10 is a schematic illustration showing a mixing chamber, automated filter and spectrophotometer, a controlled volume of titrant injected in increments to the sample while mixing; after each titrant solution injection, an aliquot of analyte is extracted from the mixing chamber through the automated filter and the filtrate's spectra absorbance measured by spectrophotometer is transmitted to computer;

FIG. 11 is a cross section of the pipeline and system in FIG. 6 showing, after quantitative volumetric titration value are determined, a controlled volume of water is injected into the mixing chamber via the automated sampler to flush out and remove the spent slurry or process water sample through a drainage port at the bottom of mixing chamber; the flushing water also cleans the sampler, the mixer impeller, the pH probe, the automated filter, and the mixing chamber interior while mixing; the online and automated analyzer is ready to analyze the next sample;

FIG. 12 is a process diagram of the automated and online mineral slurry and process quantitative volumetric titration analyzer operation.

FIG. 13 is a graph illustrating a series of filtrate spectra absorbances for methylene blue (MB) for a model clay mixture.

FIG. 14 is a graph illustrating a curve of spectra absorbance at 664 nm vs. cumulative MB volume injected for model clay mixture in FIG. 13 showing two sections of the curve can be extrapolated and the junction is the MB titration endpoint that can be used to determine the methylene blue index (MBI) value (empty circle). MBI value determined from manual titration and visual halo method according to the ASTM C837-09 is also plotted as reference (solid circle).

FIG. 15 is a schematic illustration of an embodiment of an automated online mineral slurry and process water hardness analyzer shown installed on a live slurry or process water pipeline;

FIG. 16 is a cross section of the pipeline and system in FIG. 15 showing an automated sampler taking a controlled volume of slurry or process water sample from a live pipeline (or other vessel) at controlled time intervals;

FIG. 17 is a cross section of the pipeline and system in FIG. 15 showing a controlled volume of dilution water being injected into the automated sampler to dilute the slurry or process water sample and flush it into the mixing chamber;

FIG. 18 is a schematic illustration of the chemicals and EBT containers, and mixing chamber showing a controlled volume of chemicals solution injected in increments to the diluted slurry sample while mixing, the chemicals injection continues until a target pH value is reached as measured by pH probe;

FIG. 19 is a schematic illustration showing a mixing chamber, automated filter and spectrophotometer, a controlled volume of EDTA injected in increments to the sample while mixing; after each EDTA solution injection, an aliquot of analyte is extracted from the mixing chamber through the automated filter and the filtrate's spectra absorbance measured by spectrophotometer is transmitted to computer;

FIG. 20 is a cross section of the pipeline and system in FIG. 15 showing, after the liquid hardness values are determined, a controlled volume of water is injected into the mixing chamber via the automated sampler to flush out and remove the spent slurry or process water sample through a drainage port at the bottom of mixing chamber; the flushing water also cleans the sampler, the mixer impeller, the pH probe, the automated filter, and the mixing chamber interior while mixing; the online and automated analyzer is ready to analyze the next sample;

FIG. 21 is a graph illustrating the change of filtrate spectra absorbance at 620 nm before and after the titration endpoint during water hardness measurement for an oil sands SAGD process water; The spectra absorbance at 620 nm shows a range of increase with increasing EDTA as it approaches the endpoint. The titration endpoint corresponding to spectra absorbance shifted which is an indication that EDTA molecules are complexed with the Calcium and Magnesium ions and un-complexed (released) the metal ions from the bounding with EBT, the process resulted the spectra absorbance change;

FIG. 22 is the graph of filtrate spectra absorbance plotted against the cumulative EDTA solution volume titrated for the oil sands SAGD process water in FIG. 21. There are two distinct curves before and after reaching the titration endpoint. The two curves met at a junction which indicates a titration endpoint. This junction or endpoint can be correlated by computer and to replace the visual determination by human eyes.

FIG. 23 is a series of pictures of the oil sands SAGD water illustrated in FIG. 21. As EDTA volume increased, it un-complexed (released) the Calcium and Magnesium ions from their complex (bound) with EBT. The colour change at 7.5 mL indicated the visual titration endpoint, which matches the spectra endpoint in FIGS. 21 and 22. The spectra endpoint can be automatically detected by the spectrophotometer and plotted along with other parameters to determine the liquid hardness.

FIG. 24 is a process diagram of the automated and online mineral slurry and process water hardness analyzer operation.

### DETAILED DESCRIPTION

### pH Analyzer:

In some embodiments, the present invention provides an automated and online mineral slurry and process water pH analyzer to determine liquid pH of mineral slurry or process water by withdrawing a controlled volume sample of slurry or process water from a live process, the process sample is mixed with a controlled volume and pH of dilution water in a mixing chamber, the pH of the diluted sample mixture is measured and correlated to determine the process sample's pH.

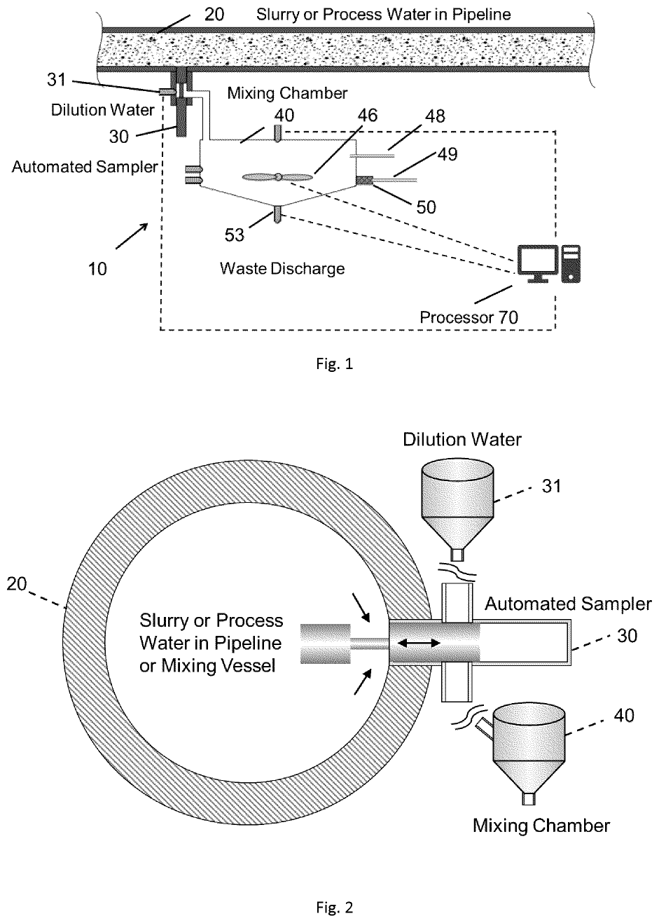

Referring to FIGS. 1-5, there is illustrated a schematic diagram of an embodiment of an automated and online mineral slurry and process water pH analyzer 10 in accordance with an embodiment of the present invention. pH analyzer 10 includes automated sampler 30 that is operably mounted on a mineral slurry or process water pipeline, vessel, tank or conduit, such as pipeline 20, to withdraw a sample of the slurry or process water from the flow without interfering with the operation of the pipeline or conduit. The automated sampler 30 withdraws a known volume of the slurry or process water and transfers it to mixing chamber 40. An example of a suitable automated sampler 30 is an ISOLOK™ automated sampler produced and distributed by Sentry Equipment of Oconomowoc, WI, USA; however, other automated samplers may be suitable for use as automated sampler 30 as would be apparent to a person skilled in the art in light of the present disclosure. For example, some automated wall samplers or isokinetic samplers may be suitable. The automated sampler is preferably coupled to and remotely actuatable and controlled by a computer, processor or other controller, herein referred to generally as processor 70.

pH analyzer 10 includes mixing chamber 40 that is downstream from and fluidly connected to the automated sampler 30. Mixing chamber 40 may include an agitator or mixer such as impeller 46 for thoroughly mixing the fluid sample. Other mixers and agitators may be used as would be apparent to a person skilled in the art in light of the present disclosure. The mixer or agitator is preferably coupled to and remotely actuatable by processor 70. Mixing chamber 40 receives the sample from the automated sampler 30 and mixes and disperses the sample by the impeller 46.

Online mineral slurry and process water pH analyzer 10 includes a source of water such as water container 31 that is fluidly connected to the automated sampler 30. The water source or water container 31 is operable to supply a controlled volume of water of a known pH to the automated sampler 30 to flush the slurry sample out of the automated sampler 30 and into the mixing chamber 40 and to dilute the sample. Preferably the water source such as water container 31 is coupled to and remotely actuatable and controlled by processor 70 to provide said controlled volume of water to the automated sampler 30.

Mixing chamber 40 includes a temperature probe to measure the temperature of the diluted sample solution. Mixing chamber 40 includes a thermal jacket connecting to a recirculating chiller operable to heat or cool the sample mixture to a desired temperature. Mixing chamber 40 includes a pH probe 48 for sensing the pH of the diluted sample mixture.

Online mineral slurry and process water pH analyzer 10 may include an automated filter 50 downstream of mixing chamber 40 and having porous filter element through which the diluted sample mixture is passed, after being processed in mixing chamber 40, to obtain the liquid analyte free of particles and hydrocarbon droplets. For example, porous filter element may comprise nylon membrane or other materials with pore size suitable for the mineral sample to be analyzed. The automated filter 50 is operable to remove coarse particulate and hydrocarbon droplets from the diluted sample mixture and allow the liquid filtrate to pass therethrough. An example of automated filter 50 may be a second ISOLOK™ automated sampler 51 of a style in which as a plunger of the sampler retracts, the front end of plunger collapses and generates pressure. This second ISOLOK automated sampler 51 may be coupled to the mixing chamber 40 so that it extracts an aliquot of the diluted sample mixture from the mixing chamber, and that is further coupled to one or more filter media or element 52. Once the aliquot of the diluted sample mixture is extracted from the mixing chamber, as the plunger of the sampler device retracts the front end of plunger collapses and generates pressure to propel the aliquot against a filter media to generate filtrate. The system may be configured to automatically replace the filter media with fresh filter media when it has become fouled. For example, automated filter may have an automated filter changer composed of multiple syringe filters 52, which connected to the outlet of the second ISOLOK™ automated sampler 51. When the processor 70 detects that the pressure resistance of the filter element has reached a threshold point due to fouling, it may provide instructions to the automated filter changer 52 to switch to a fresh filter element. While the foregoing is an example of an automated filter 50 and automated filter changer 52, other embodiments of an automated filter may be used that can extract multiple aliquots, filter them into filtrates. The pH of filtrate is measured by pH probe 49.

Online mineral slurry and process water pH analyzer 10 may include an oil skimming plate 41 installed in the mixing chamber 40 above the automated filter 50 to minimize oil or bitumen droplets entering the automated filter 50. A similar oil skimming plate may be provided above the pH probe 48 in the mixing chamber 40 to minimize oil or bitumen droplets from the sample attach to the pH probe 48.

pH probes 48 and 49 may be of a conventional type known in the art, such as for example a model PHCN-37 pH Controller and PHE-7352-15 pH probe manufactured and distributed by Omega. However, this example is for illustrative purposes and it would be apparent to a person skilled in the art in light of the present disclosure that other pH probes may be suitable for use as pH probes 48 and 49 in the present invention. pH probes 48 and 49 are coupled to processor 70 and provides the measured pH values to processor 70.

In operation, processor 70 instructs the automated sampler 30 to take a slurry or process water sample from a live pipeline or mixing vessel 20. Processor 70 then instructs the water source such as water container 31 to inject a controlled volume and known pH of dilution water into the automated sample 31 to flush the sample out of the automated sampler and thereby effect its dilution and transfer into the mixing chamber 40.

The processor 70 activates the mixer such as impeller 46 to disperse the diluted sample in the mixing chamber 40. After a predetermined time suitable for adequately dispersing the diluted sample, the processor 70 obtains a pH measurement of the diluted sample mixture from the pH probe 48.

In some aspects, processor 70 may instructs automated filter 50 to withdraw an aliquot of the dilute sample mixture. The withdrawn aliquot is filtered by automated filter 50 and the filtrate is measured by pH probe 49. Processor 70 may be operable to provide instructions to the automated filter 50. Processor 70 correlates the measured pH of the dilute sample mixture to the pH of process sample as further described herein. If the process sample is a slurry, the liquid content of the slurry is obtained from the slurry density measured by the densitometer installed near the pH analyzer.

The pH analyzer 10 provides a new pH measurement method and apparatus to determine process pH where direct measurement of pH from the process is not feasible due to hydrocarbons and abrasive solid particles contained in the process sample. This is achieved by obtaining a controlled volume of process sample, with an unknown pH, diluting it with a controlled volume of diluting water having a known pH, followed by measuring the pH of the diluted mixture. In some instances, the process sample may be diluted five or more times such that the hydrocarbons contained in the sample are much less likely to coat the pH probe 48 and solid particles in the diluted sample are much less likely to cause erosion as they are not flowing past the pH probe 48 at as high of a velocity in the mixing chamber as in the pipeline 20. In some instances, an aliquot of process sample may be extracted through the automated filter 50 to remove particles and hydrocarbons and the pH of filtrate is measured by the pH probe 49. In addition, the mixing chamber, pH probes and accessories are automatically cleaned after each measurement, there is no buildup of hydrocarbon coating on the pH probes. The pH of the extracted process sample can be calculated using the known volumes of liquid in the process sample, dilution water and diluted mixture, the known pH of the dilution water and the measured pH of the diluted sample mixture through [OH⁻] ion concentration conversion between diluted mixture, dilution water and process sample.

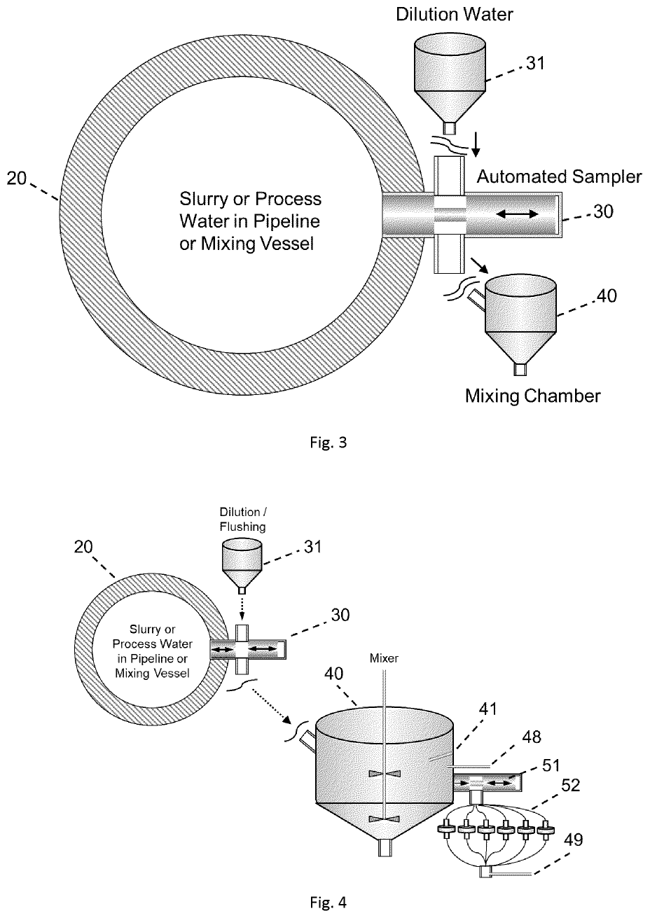

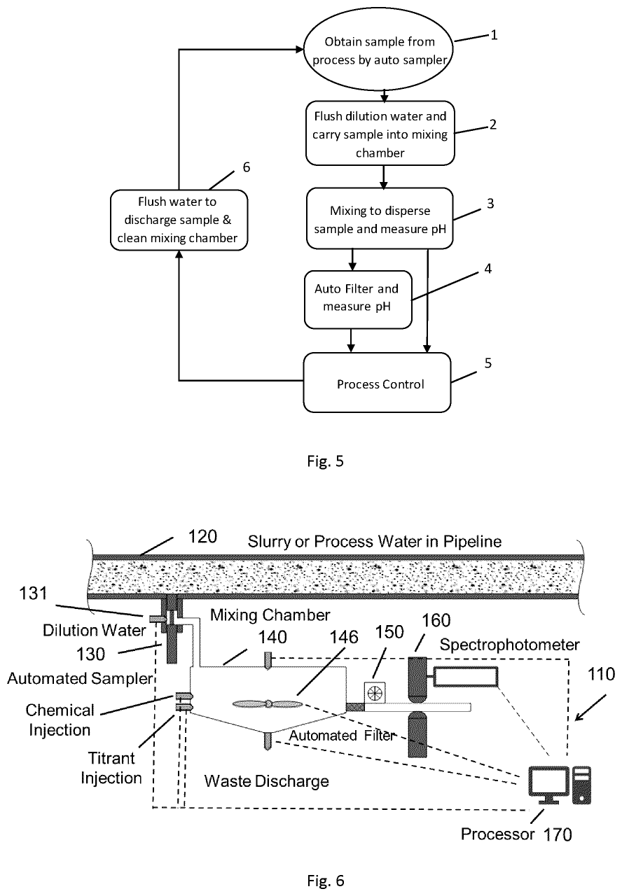

With reference to the numbered analysis steps in FIG. 5:
At step 1 the automated sampler 30 takes a controlled volume of slurry or process water sample from a live pipeline or mixing vessel 20 upon instructions communicated from processor 70 (FIG. 2). The samples may be taken at controlled time intervals or as desirable to enable meaningful process control.
At step 2, upon instructions from the processor 70, a controlled volume of dilution water with known pH is injected by the water container 31 into the automated sampler 30 that flushes the slurry or process water sample into the mixing chamber 40 (FIG. 3). The dilution water may be dispensed by a pump controlled by the processor 70.
At step 3 the mixing chamber 40 is equipped with a mixer such as impeller 46 and a pH probe 48. The impeller 46 is activated by the processor 70 to disperse solid particles in the diluted sample mixture. The pH of diluted sample mixture is measured by the pH probe 48 and the measurement is communicated to the processor 70, which determines the pH of process sample.

In some instances and at step 4, an aliquot of the process sample in mixing chamber 40 is filtered by the automated filter 50 and the pH of filtrate is measured by the pH probe 49 and the measurement is communicated to the processor 70, which determines the pH of process sample.
At step 5, after the sample pH value is determined, the processor 70 instructs the water container 31 to inject a volume of water into the mixing chamber via the automated sampler to flush the automated sampler 30 and the mixing chamber 40 to remove the spent slurry or process water. The processor causes a drainage port 53 at the bottom of mixing chamber 40 to open to allow the expulsion of the spent sample. The flushing water also cleans the automated sampler 30, the mixer 46 and pH probe 48, and the interior of the mixing chamber by engaging the mixer 46. Thereafter the pH analyzer 10 is ready to analyze the next sample.

The pH of diluted mixture as measured by pH probe 48 and/or pH probe 49 is correlated to the pH of process sample as follows.

pH is a measurement of acidity or alkalinity of a liquid solution, which is determined by the relative number of hydrogen ions [H⁺] and pOH is determined by hydroxyl ions [OH⁻] present in the solution. pH and pOH are defined by the following equations:

pH=−log₁₀[H⁺] 1

pOH=−log₁₀[OH⁻] 2

pH+pOH=14 3

The following equations can be derived from Equations 1 to 3:

[H⁺]=10^{-pH} 4

[OH⁻]=10^{-pOH}=10^{(pH-14)} 5

[H⁺]+[OH⁻]=1×10⁻¹⁴ 6

For a controlled volume of liquid sample withdraw from the process, its pH₁ is unknown but can be determined from [OH⁻]₁ which has the unit of mol/L:

pH₁=14−pOH₁=14+log₁₀[OH⁻]₁ 7

V₁=known volume 8

[OH⁻]₁ can be determined from the following procedures. By mixing a controlled volume V₁ of process sample with a controlled volume of dilution water V₂ at known pH₂, the [OH⁻]₁ can be determined from pH₂ using the following equations:

[OH⁻]₂=1×10^{(pH}^{₂}⁻¹⁴⁾ 9

V₂=known volume 10

After mixing the process sample with the dilution water, the combined mixture pH₃ can be measured and [OH]₃ is determined by:

[OH⁻]₃=1 ×10^{(pH}^{₃}⁻¹⁴⁾ 11

Therefore, the unknown pH₁ can be solved from Equations 7 and 12, with conversions of [OH⁻] concentration from mol/L to mol using equation 13:

[OH⁻]₁=[OH⁻]₃−[OH⁻]₂ 12

V₃=V₁+V₂ 13

There will be complications from buffering effect of other ions present in the process sample and effects such as temperatures, but these effects can be pre-determined through calibration.

### Example 1

Four oil sands tailings samples were tested using an automated and on-line pH analyzer described herein. The process sample volume V₁, dilution water volume V₂ and the dilution water pH₂ are controlled; the combined mixture volume V₃ are known; the combined mixture pH₃ is measured by pH probe 48. The process sample pH₁ can be correlated from the controlled and measured values. The difference between correlated pH₁ and the actual pH₁ is within 10% for the four oil sands tailings samples. The difference can be further reduced through equipment and procedure optimizations.

TABLE 1
Examples of On-line pH Meter Measurements on Oil Sands Process Water Samples

| | | | | | | | | | | Measured | Difference |
|---|---|---|---|---|---|---|---|---|---|---|---|
| Oil Sands | Dilution Water | | | Combined Mixture | | | Correlated Process Sample | | | Process | Correl. |
| Sample V₁ | pH₂ | V₂ | [OH⁻]₂ | pH₃ | V₃ | [OH⁻]₃ | [OH⁻]₁ | [OH⁻]₁ | pH₁ | pH₁ | vs. Meas. |
| (mL) | (pH) | (mL) | (mol) | (pH) | (mL) | (mol) | (mol) | (mol/L) | (pH) | (pH) | (%) |
| 5.30 | 6.38 | 449.7 | 1.08E−08 | 7.28 | 455.0 | 8.67E−08 | 7.59E−08 | 1.43E−05 | 9.16 | 8.84 | 3.6 |
| 15.80 | 6.38 | 449.6 | 1.08E−08 | 7.80 | 465.4 | 2.94E−07 | 2.83E−07 | 1.79E−05 | 9.25 | 8.86 | 4.4 |
| 5.50 | 6.38 | 199.9 | 4.79E−09 | 7.70 | 205.4 | 1.03E−07 | 9.81E−08 | 1.78E−05 | 9.25 | 8.79 | 5.2 |
| 16.20 | 6.38 | 200.0 | 4.80E−09 | 7.90 | 216.2 | 1.72E−07 | 1.67E−07 | 1.03E−05 | 9.01 | 8.67 | 4.0 |

Some general implementations of the present invention may be in applications where direct measurement of pH from process fluid is not feasible, such as for example when hydrocarbons in the fluid may interfere the reading or even coat the pH probe and make the measurement inaccurate or impossible, and/or when solid particles in the slurry would be abrasive to the surface of pH that damage the probe in a short time.

Quantitative Volumetric Titrations

In some embodiments, the present invention provides an automated online mineral slurry and process water quantitative volumetric titration analyzer that automatically performs titrations on a sample of mineral slurry or process water and determines an endpoint of the titration based on changes in liquid spectra and correlates the endpoint to the cumulative titrant volume and one or more parameters of the mineral slurry or process water. A controlled volume of mineral slurry or process water sample is automatically withdrawn from the process and mixed with dilution water at controlled volume. The diluted mixture sample is then conditioned with chemicals and indicator and/or has its temperature regulated, as required by the titration protocol, followed by injecting in increments a controlled volume of a titrant solution. After each titrant solution injection, a filtrate is extracted from the mixture. The filtrate spectra absorbance is measured by a spectrophotometer and correlated to the cumulative volume of titrant solution and/or a parameter of the mineral slurry or process water.

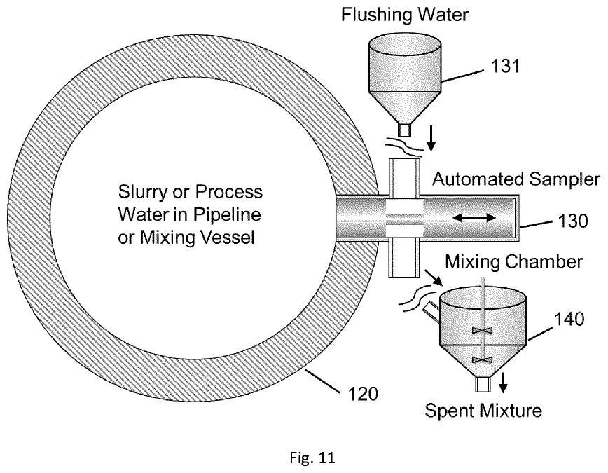

Referring to FIGS. 6-12, there is illustrated a schematic diagram of an embodiment of an automated and online mineral slurry and process water quantitative volumetric titration analyzer 110 of the present invention.

Quantitative volumetric titration analyzer 110 includes automated sampler 130 that is operably mounted on a mineral slurry or process water pipeline, vessel, tank or conduit, such as pipeline 120, to withdraw a sample of the slurry or process water from the flow without interfering with the operation of the pipeline or conduit. The automated sampler 130 withdraws a controlled volume of the slurry or process water and transfers it to mixing chamber 140. An example of a suitable automated sampler 130 is an ISOLOK™ automated sampler produced and distributed by Sentry Equipment of Oconomowoc, WI, USA; however, other automated samplers may be suitable for use as automated sampler 130 as would be apparent to persons skilled in the art in light of the present disclosure. For example, some automated wall samplers or isokinetic samplers may be suitable. The automated sampler is preferably coupled to and remotely actuatable and controlled by a computer, processor or other controller, herein referred to generally as processor 170.

The volumetric titration analyzer 110 includes mixing chamber 140 that is downstream from and fluidly connected to the automated sampler 130. Mixing chamber 140 may include an agitator or mixer such as impeller 146 for thoroughly mixing the fluid sample. Other mixers and agitators may be used as would be apparent to a person skilled in the art. The mixer or agitator is preferably coupled to and remotely actuatable by processor 170. Mixing chamber 140 receives the sample from the automated sampler 130 and mixes and disperses the sample by impeller 146.

Volumetric titration analyzer 110 includes a source of water such as water container 131 that is fluidly connected to the automated sampler 130. The water source or water container 131 is operable to supply a controlled volume of water to the automated sampler 130 to flush the slurry sample out of the automated sampler 130 and into the mixing chamber 140 and to dilute the sample. Preferably the water source 131 is coupled to and remotely actuatable and controlled by processor 170 to provide said controlled volume of water to the automated sampler 130.

Volumetric titration analyzer 110 includes one or more sources of chemicals such as chemicals containers 133 and 134 that are fluidly connected to the mixing chamber 140. The source of chemicals is operable to supply a controlled volume of chemicals to the mixing chamber 140. Preferably the source of chemicals such as chemicals containers 133 and 134 are coupled to and remotely actuatable and controlled by the processor 170 to provide said controlled volume of chemicals to the mixing chamber 140. Processor 170 instructs the source of chemicals such as chemicals containers 133 and 134 to inject a controlled volume of chemicals into the mixing chamber 140. The processor 170 instructs the mixer such as impeller 146 to disperse the diluted sample in the mixing chamber 140 to produce a diluted conditioned sample mixture.

The chemicals used in the process will vary depending on operational factors, including but not limited to the particular protocol for the volumetric titration being used, the source of the mineral slurry, and the kinds of chemicals used and quantities would be apparent to those skilled in the specific field. By way of example only, the chemicals may include acids, bases or buffers to adjust the pH of the sample, and/or chemicals to remove hydrocarbons from the slurry or process water sample, and/or chemical indicator for complexometric titration.

Mixing chamber 140 may include a temperature probe to measure the temperature of the diluted sample solution. Mixing chamber 140 may include a thermal jacket connecting to a recirculating chiller operable to heat or cool the sample mixture to a desired temperature. The temperature probe and the recirculating chiller may be each coupled to the processor 170 which is operable to compare a measured temperature value from the temperature probe to a desired temperature for the titration protocol, and to activate the recirculating chiller as required to achieve the desired temperature in the sample mixture.

Mixing chamber 140 may include a pH probe 148 for sensing a pH of the sample mixture. The pH probe 148 may be coupled to the processor 170 to provide measured values to the processor. The processor 170 may be operable to compare a measured pH value from the pH probe 148 to a desired pH value for the titration, and to activate the chemical containers 133 and/or 134 to dispense a volume of chemicals into the sample solution to achieve the desired pH value. Hence the pH probe 148 may be coupled to a feedback mechanism for regulating the volume of chemicals dispensed into the mixing chamber 140 from the source of chemicals.

Volumetric titration analyzer 110 includes a source of titrant solution such as titrant solution container 137 that is fluidly connected to the mixing chamber 140 to supply a controlled volume of the titrant solution to the mixing chamber 140. Preferably the source of titrant such as titrant solution container 137 is coupled to and remotely actuatable and controlled by the processor 170 to provide said controlled volume of titrant to the mixing chamber 140. Processor 170 instructs the source of titrant solution such as titrant solution container 137 to inject a controlled volume of titrant solution into the mixing chamber 140 at multiple times.

The titrant solution used in a quantitative volumetric titration is one that binds to a specific target compound in the diluted sample mixture to effect a change in the intensity and/or the color of the solution, and which change can be measure by a spectrophotometer. Examples of titrants include, but are not limited to:
Methylene blue (MB) as an indicator for determining the methylene blue index (MBI) value and active clay content of mineral and mineral slurry.
Phthalein Purple for determining Calcium and/or Magnesium ions at two distinctive pH ranges in a mineral slurry or process water, and/or vice versa to determine sample pH if the Calcium and/or Magnesium content in the sample is known.
A specific complexometric titration using Ethylenediamine Tetraacetic Acid (EDTA) as titrant for determining the total liquid hardness (Calcium and Magnesium combined) and/or liquid hardness contributed by Calcium or Magnesium individually of the sample slurry or process water.
Any type of Complexometric Titration that a colored complex is formed during the titration such that the endpoint of the volumetric titration is indicated by the liquid color change which can be detected by a spectrophotometer, the complexometric titration endpoint can be determined and quantified by correlating the liquid spectra absorbance and the cumulative volume of the titrant solution.

A person skilled in the art, in light of the present disclosure, would understand that other titrants may be used with the quantitative volumetric titration analyzer of the present invention to determine a parameter of the mineral slurry or process water for which such titrant is suitable using titration techniques.

Accordingly, mixing chamber 140 is operable to receive the diluted sample from the automated sampler 130, a controlled volume of chemicals from the chemicals containers 133 and 134, controlled volumes of titrant solution from the titrant solution container 137, and to thoroughly mix these compounds into a diluted sample mixture.

Volumetric titration analyzer 110 includes an automated filter 150 downstream of mixing chamber 140 and having porous filter element through which the diluted sample mixture is passed, after being processed in mixing chamber 140, to obtain the liquid analyte. For example, porous filter element may comprise nylon membrane or other materials with pore size suitable for the mineral sample to be analyzed. The automated filter 150 is operable to remove coarse particulate and hydrocarbon droplets from the diluted sample mixture and allow the liquid filtrate to pass therethrough. An example of automated filter 150 may be a second ISOLOK™ automated sampler 151 of a style in which as a plunger of the sampler retracts, the front end of plunger collapses and generates pressure. This second ISOLOK automated sampler 151 may be coupled to the mixing chamber 140 so that it extracts an aliquot of the chemically treated and diluted sample mixture from the mixing chamber, and that is further coupled to a filter changer 152. Once the aliquot of the chemically treated sample mixture is extracted from the mixing chamber, the plunger of the sampler device retracts that the front end of plunger collapses and generates pressure to propel the aliquot against a filter media to generate filtrate. The system may be configured to automatically replace the filter media with fresh filter media when it has become fouled. For example, the automated filter changer 152 connects to the outlet of the second ISOLOK™ automated sampler 151. When the processor 170 detects that the pressure resistance of filter element has reached a threshold point due to fouling, it may provide instructions to the automated filter changer 152 to switch to a fresh filter element. While the foregoing is an example of an automated filter 150, other embodiments of an automated filter may be used that can extract multiple aliquots, filter them into filtrates and convey the filtrates to the spectrophotometer.

Volumetric titration analyzer 110 may include an oil skimming plate 141 installed in the mixing chamber 140 above the automated filter 150 to minimize oil or bitumen droplets entering the automated filter 150. A similar oil skimming plate may be provided above the pH probe 148 in the mixing chamber 140 to minimize oil or bitumen droplets from the sample attach to the pH probe 148.

Volumetric titration analyzer 110 includes a spectrophotometer 160 having an optical flowcell that receives the filtrate from the automated filter 150. Spectrophotometer 160 is operable to measure the spectra absorbance of the filtrate in the flowcell at pre-calibrated range of wavelengths, and the spectra absorbance is transmitted to processor 170 for computational analysis. A suitable spectrophotometer 160 for use in the present invention includes but is not limited to a Model CXR-25 Black Comet Spectrophotometer manufactured by Stellar Net USA. A suitable flowcell for use in the present invention includes but is not limited to a Model RK-83057-79 manufactured by Cole-Parmer. The spectra absorbance data is used by the processor 170 to determine a parameter of the slurry or process water sample, which may be used on its own or in conjunction with other parameters to control the process either upstream or downstream of the automated sampler 130.

Accordingly, the diluted sample mixture in the mixing chamber 140 is conditioned with chemicals from the chemical containers 133 and 134 until, for example, the diluted sample mixture reaches a target pH as measured by the pH probe 148. While mixing, a controlled volume of titrant solution is injected in increments under the control of processor 170 into the sample mixture from the titrant solution container 137. After each titrant solution injection, a small aliquot of the sample mixture is withdrawn from the mixing chamber through automated filter 150. Processor 170 instructs automated filter 150 to withdraw an aliquot of the dilute sample mixture after an injection of the titrant solution and once sufficient time has elapsed to enable thorough mixing of the sample mixture and titrant. The withdrawn aliquot is filtered by automated filter 150 and the filtrate is transferred, as a result of pressure generated by the automated filter 150 or by a peristatic pump, to the spectrophotometer 160 via an optical flowcell where the spectra absorbance of the filtrate is measured. Processor 170 may be operable to provide instructions to the automated filter 150. Processor 170 may be coupled to the spectrophotometer 160 to receive spectra absorbance measurements and to store such data in memory. Processor 170 may be operable to analyze the stored spectra data to determine a titration endpoint, and to correlate the titration endpoint and the cumulative titrant volume injected with the desired parameter to be determined for the sample.

The injection of titrant solution and the spectra absorbance measurement of each aliquot taken after each such titrant solution injection continues until the processor determines that the measured spectra absorbance data indicates that an endpoint in the titration has been reached or passed, which indicates total reaction of the titrant solution with the target ion(s) in the liquid from which a desired parameter of the liquid may be determined, or until enough spectra absorbance data is obtained to be useful in deriving a desired parameter of the sample. The spectra absorbance and the cumulative titrant solution volume injected can be correlated by the processor to determine the desired parameter, which can be used to achieve effective process control and management.

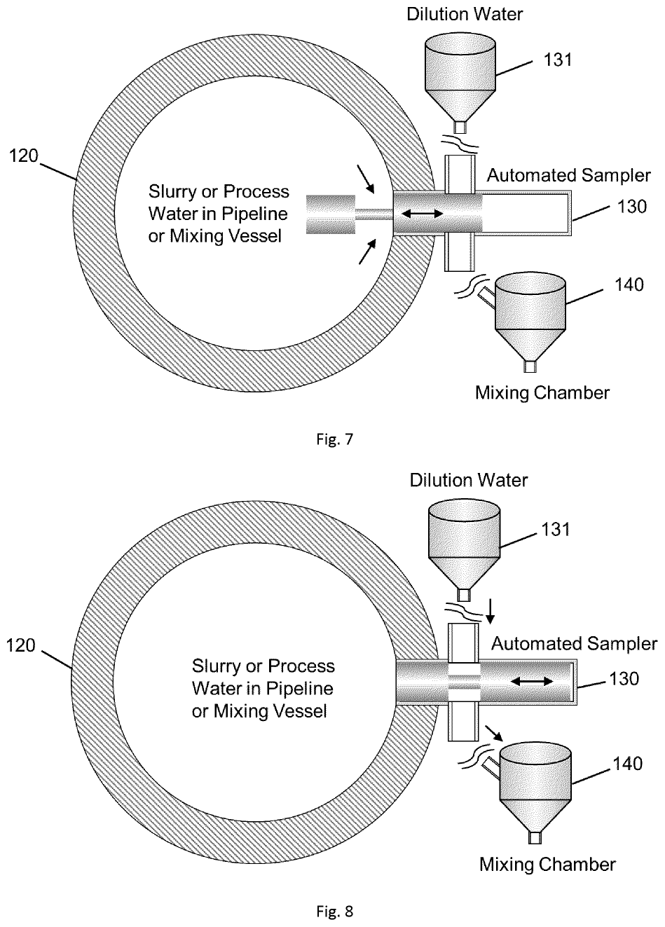

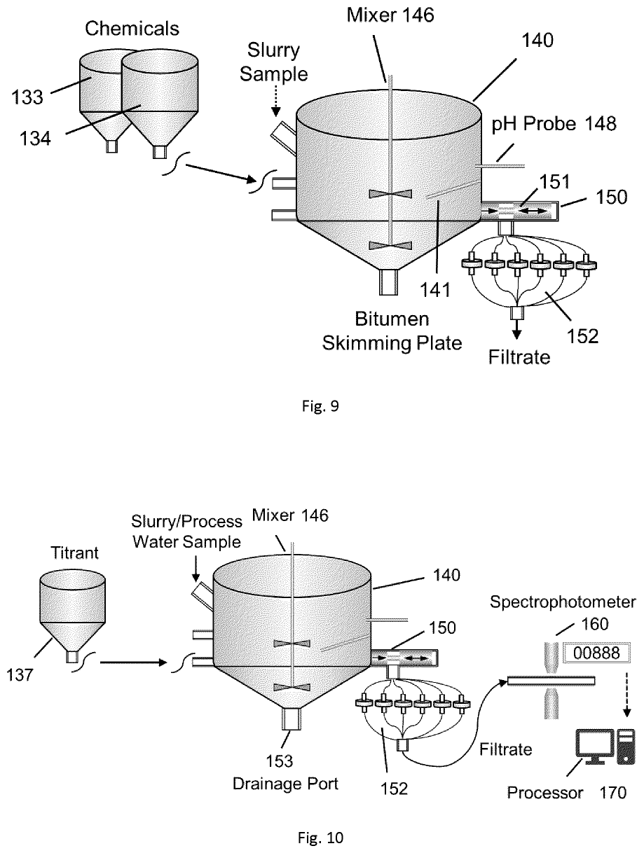

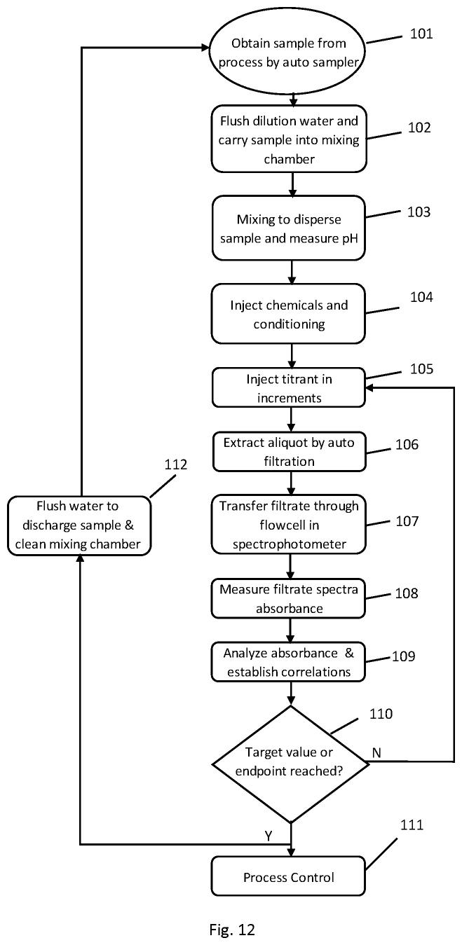

More specifically, with reference to the numbered analysis steps in FIG. 12:
At step 101, the automated sampler 130 takes a controlled volume of mineral slurry or process water sample from a pipeline, container or vessel such as pipeline 120 pursuant to instructions received from the processor 170. (FIG. 6). Processor 170 may be programmed to provide instructions to the automated sampler 130 to obtain a sample at certain times, time intervals, or based on other parameters.
At step 102, upon instructions from the processor 170, a controlled volume of dilution water is injected by the water container 131 into the automated sampler 130 that flushes the sample into the mixing chamber 140 (FIG. 7). The dilution water may be dispensed by a pump controlled by the processor 170.
At step 103 the mixing chamber 140 is equipped with a mixer such as impeller 146 and a pH probe 148. The impeller 146 is activated by the processor 170 to disperse solid particles in the diluted sample mixture and enhance reactions.
At step 104, while mixing, a controlled volume of chemical solutions from the chemical solutions containers 133 and 134 may be injected in increments to the diluted sample until a target pH value is reached as measured by the pH probe 148. The chemicals injection volume is controlled by the processor 170 which take into consideration the pH measurement (FIG. 9). The chemical solutions may be dispensed by dispenser pumps controlled by the processor 170.
At step 105 while mixing, a controlled volume of titrant solution is injected in increments from the titrant solution container 137 to the chemically conditioned sample mixture (FIG. 10). The titrant solution may be dispensed by a dispenser pump controlled by the processor 170.
At step 106, after each titrant solution injection, an aliquot of analyte is extracted from the mixing chamber and through the automated filter 150.
At step 107, the filtrate analyte is transferred through an optical flowcell of spectrophotometer 160 by the pressure from the automated filter 150 or by a peristaltic pump.
At step 108, the filtrate is measured by the spectrophotometer 160 at pre-calibrated wavelength range(s) and the measured spectra absorbance data is transmitted to the processor 170.
At step 109, the processor is operable to analyze the spectra absorbance data from the spectrophotometer 160 to determine if an endpoint in the titration has been reached and to determine the endpoint values and the cumulative titrant volume injected.
At step 110, if an endpoint has not been reached, steps 105-109 are repeated until a target spectra absorbance value (endpoint) is reached, which indicates a completion of the titration, or the endpoint is exceeded, or enough spectra absorbance data is generated to enable correlation to the desired parameter(s) based on pre-calibration data.
Also at step 110, the spectra absorbance value and injected titrant solution cumulative volume are used to correlate and determine the desired parameter. Other slurry or process water properties may be measured by other instruments installed on the system, and these values may also be factored into the determination of the desired parameter in accordance with titration protocols and standards.
At step 111, the value of the desired parameter is used in a feedback or feed forward systems for controlling the process parameters.
At step 112, after the desired parameter values are determined, a controlled volume of water from the water source such as water container 131 is injected into the mixing chamber 140 via the automated sampler 130 to flush out the spent sample through a drainage port 153 at the bottom of mixing chamber 140. The processor 170 is operable to instruct the water source to inject the volume of water at an appropriate time, such as when the titration is complete, to activate the mixer 146 during or after the water injection, and to open drainage port 153 to allow the water and spent sample to be expelled from the mixing chamber 140. The processor may be operable to provide multiple flushing instructions to the water source to achieve complete flushing of the automated sampler 130, the mixing chamber 140, and the other parts and probes within the mixing chamber. Thus, the flushing water cleans the automated sampler, the mixer impeller and pH probe, the automated filter, and the mixing chamber interior by engaging the mixer.
After step 112, the volumetric titration analyzer is ready to analyze the next sample by starting at step 101.

Volumetric titration analyzer 110 provides a new method and apparatus that utilizes a spectrophotometer to automatically determine a titration endpoint as a replacement of a conventional titration procedure. This is achieved by a series of online and automated procedures, including automated withdrawal of samples from a live process, condition the sample in a mixing chamber, incrementally adding a controlled volume of the titrant solution, extracting a filtrate by an automated filter, measuring the filtrate spectra absorbance by a spectrometer, and using the spectra absorbance measurements to determine when an endpoint of the titration has been reached and correlating the spectra absorbance data with cumulative titrant solution volume injected to reach the titration endpoint to determine a parameter of the mineral slurry or process water. The volumetric titration analyzer can be used automatically and continuously, and it improves the accuracy by eliminating human subjective and visual endpoint detection. Furthermore, the spectra absorbance data, even before reaching the endpoint, can be used for correlation and process control, which can replace or supplement the endpoint detection and shorten the measuring time.

In an embodiment, the quantitative volumetric titration analyzer of the present invention is configured to determine the active clay content of a mineral slurry sample. The titrant solution is methylene blue (MB). At step 109, the absorbance spectra measured by the spectrophotometer 160 is analyzed by the processor 170 to determine a titration endpoint. The volume of MB solution used to reach the titration endpoint, along with normality (concentration) of the MB solution and sample mass, can be used to determine methylene blue index (MBI) value of the mineral sample based on the following known equation:

$$M B I = \frac{E \times V}{W} \times 100 \qquad 14$$

Where:
MBI=methylene blue index for the mineral sample in meq/100 g;
E=milliequivalents of methylene blue per millilitre;
V=millilitres of methylene blue solution required for the titration; and
W=grams of mineral sample, dry basis.

There are many empirical MBI equations derived from Equation 14 to be applied for specific minerals such as, for example, oil sands tailings.

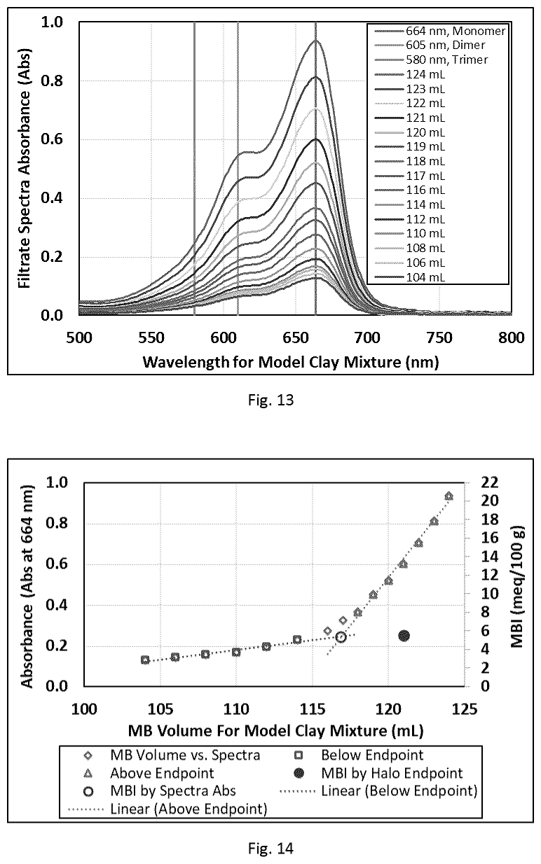

Referring to FIG. 13, there is shown a series filtrate spectra absorbances measured by spectrophotometer in accordance with the present invention for MB treated model clay mixtures composed of kaolinite (non-active clay), sodium bentonite (active clay) and silica flour (silica). FIG. 13 shows a series filtrate spectra absorbances vs. a range of wavelengths as a function of cumulative MB volumes injected, increasing the MB injection volume increases the spectra absorbance until passing the titration endpoint. Also showing in FIG. 13 are specific wavelengths corresponding to MB sub-compounds such as monomer (MB′ at 664 nm), dimer ((MB⁺)₂ at 610 nm) and trimer ((MB⁺)₃ at 580 nm). Absorbance at 664 nm (monomer) has the most sensitive peak for this particular clay mixture, but other peaks and dips (e.g., 610 nm dimer) provide useful information about clay surfaces and interlayers. Referring to FIG. 14, there is shown a graph illustrating a curve of filtrate spectra absorbance at 664 nm as a function of cumulative MB volume injected for the same model clay mixture in FIG. 13. The filtrate spectra absorbance shows two distinctive curves, each can be extrapolated and cross at a junction that is the titration endpoint. This endpoint from the spectra absorbance curves can be used to determine the MBI value (empty circle). It can replace the prior art visually determined titration endpoint (solid circle) from halo identification method based on ASTM C837-09. The endpoint determined by either spectra absorbance or halo identification is an indication that the clay edges, external surfaces and interlayers being adsorbed by the dye MB molecules such that free dye MB molecules remain in solution and thereby cause an increase in spectra absorbance or appear as a halo around the droplet on filter paper in the ASTM C837-09 method.

Water Hardness:

In some embodiments, the present invention provides an automated and online mineral slurry and process water hardness analyzer to determine the hardness of a mineral slurry or process water by withdrawing a controlled volume of slurry or process water sample from a live process, the process sample is mixed with a controlled volume of dilution water in a mixing chamber, the pH of the diluted mixture is measured. The diluted mixture is conditioned with chemicals to reach a target pH. A controlled dose of water hardness indicator such as Eriochrome Black T (EBT) is injected into the mixture, followed by injecting water hardness titrant such as Ethylenediamine Tetraacetate Acid (EDTA) in increments; at each EDTA injection, a filtrate is extracted from the mixture and analyzed by a spectrophotometer. The filtrate's spectra absorbance is used to determine the EDTA titration endpoint and correlate to the hardness of liquid. The analyzer can be installed on a live slurry conduit or water supply line and can automatically and continuously take and analyze slurry or process water samples.

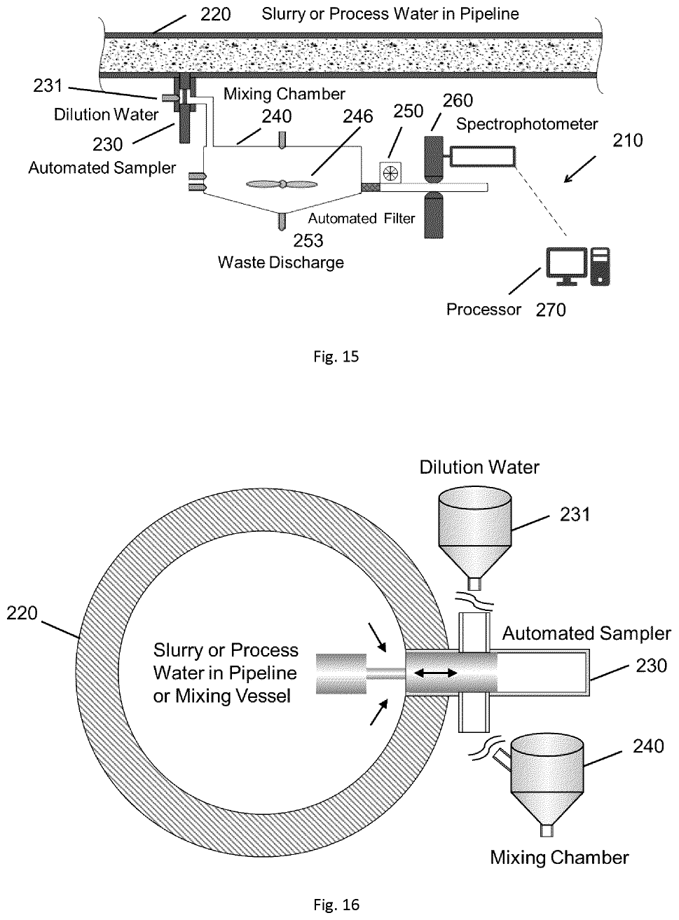

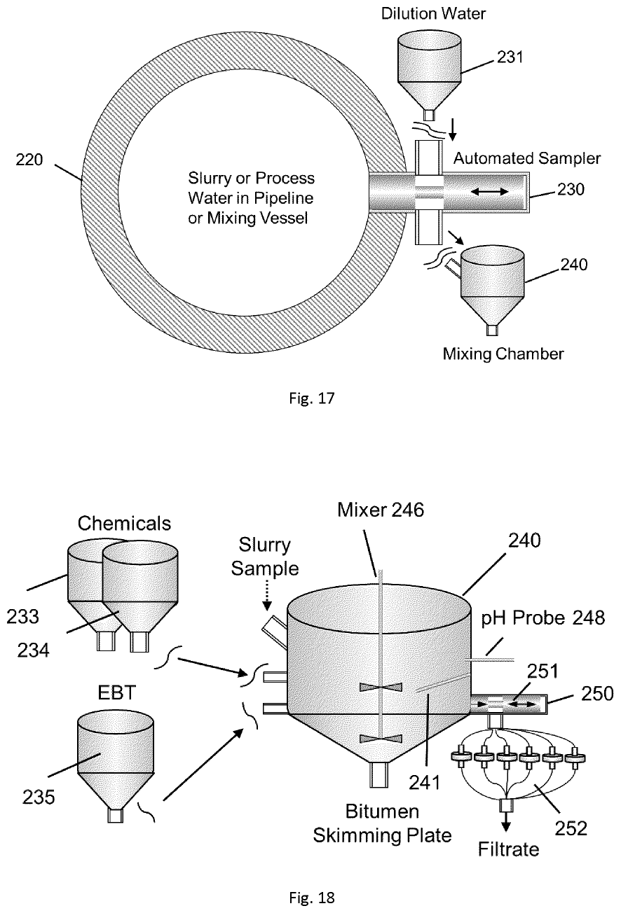

Referring to FIGS. 15-24, there is illustrated an embodiment of an automated and online mineral slurry and process water hardness analyzer 210 of the present invention.

Liquid hardness analyzer 210 includes automated sampler 230 that is operably mounted on a mineral slurry or process water pipeline, vessel, tank or conduit, such as pipeline 220, to withdraw a sample of the slurry or process water from the flow without interfering with the operation of the pipeline or conduit. The automated sampler 230 withdraws a set volume of the slurry or process water and transfers it to mixing chamber 240. The automated sampler is preferably remotely actuatable and controlled by a computer or other programmable controller. An example of a suitable automated sampler 230 is an ISOLOK™ automated sampler produced and distributed by Sentry Equipment of Oconomowoc, WI, USA; however, other automated samplers may be suitable for use as automated sampler 230 used as would be apparent to persons skilled in the art in light of the present disclosure. For example, some automated wall samplers or isokinetic samplers may be suitable. The automated sampler is preferably coupled to and remotely actuatable and controlled by a computer, processor, or other controller, herein referred to generally as processor 270.

Liquid hardness analyzer 210 includes mixing chamber 240 that is downstream from and fluidly connected to the automated sampler 230. Mixing chamber 240 may include an agitator such as impeller 246 for thoroughly mixing the fluid sample. Other mixers and agitators may be used as would be apparent to a person skilled in the art. The agitator or agitators are preferably remotely actuatable and controlled by a computer or other programmable controller. Mixing chamber 240 receives the slurry sample from the automated sampler 230 and mixes and disperses the sample.

Liquid hardness analyzer 210 includes a source of water such as water container 231 that is fluidly connected to the automated sampler 230. The water source is operable to supply a controlled volume of water to the automated sampler 230 to flush the slurry sample out of the automated sampler 230 and into the mixing chamber 240 and to dilute the sample. Preferably the water source is remotely actuatable and controlled by a computer or other programmable controller to provide said controlled volume of water to the automated sampler 230.

Liquid hardness analyzer 210 includes one or more sources of chemicals such as chemicals containers 233 and 234 that are fluidly connected to the mixing chamber 240. The source of chemicals is operable to supply a controlled volume of chemicals to the mixing chamber 240. Preferably the source of chemicals is remotely actuatable and controlled by a computer or other programmable controller to provide said controlled volume of chemicals to the mixing chamber 240. The chemicals used in the process will vary depending on operational factors, including but not limited to the source of the mineral slurry, and the kinds of chemicals used and quantities would be apparent to those skilled in the specific field. By way of example only, the chemicals may include acids, bases or buffers to adjust the pH of the sample, and/or chemicals to remove hydrocarbons from the slurry or process water sample.

Liquid hardness analyzer 210 includes a source of water hardness indicator that is fluidly connected to the mixing chamber 240 to supply a controlled volume of the indicator to the mixing chamber 240. Preferably the source of indicator is remotely actuatable and controlled by a computer or other programmable controller to provide said controlled volume of indicator to the mixing chamber 240. A preferred water hardness indicator is Eriochrome Black T (EBT), or other liquid hardness indicators such as hydroxy naphthol blue, and an example of a source of indicator is EBT container 235 (FIG. 18).

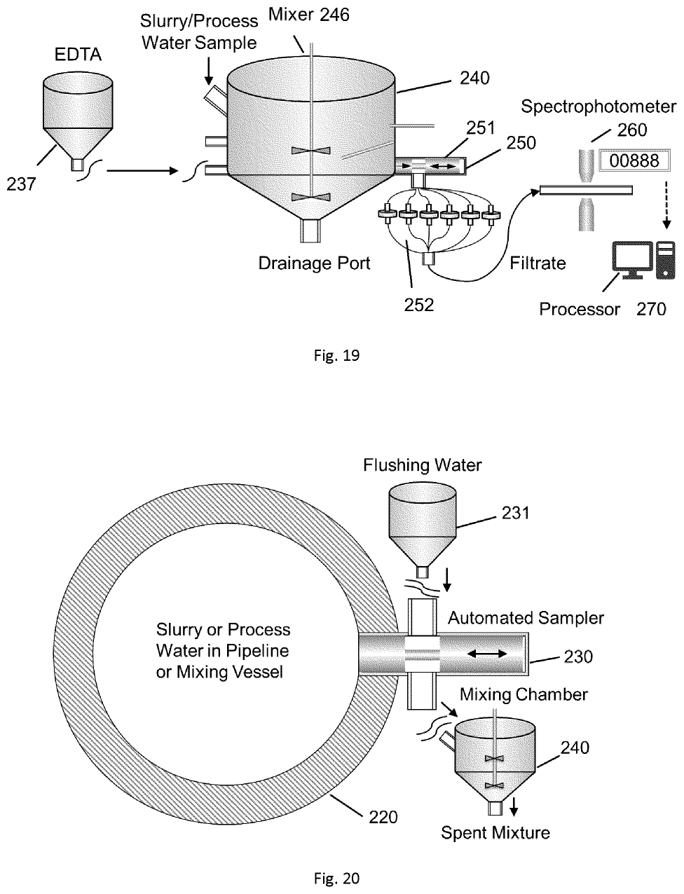

Liquid hardness analyzer 210 also includes a source of water hardness measurement solution that is fluidly connected to the mixing chamber 240 to supply a controlled volume of measurement solution to the mixing chamber 240. Preferably the source of measurement solution is remotely actuatable and controlled by a computer or other programmable controller to provide said controlled volume of measurement solution to the mixing chamber 240. A preferred water hardness titrant is Ethylenediamine Tetraacetate Acid (EDTA), or other water hardness titrant such as Phthalein Purple, and an example of a source of titrant is EDTA container 237 (FIG. 19).

Accordingly, mixing chamber 240 is operable to receive the diluted slurry sample from the automated sampler 230, a controlled volume of chemicals from the chemicals containers 233 and 234, a controlled volume of liquid hardness indicator EBT from the EBT container 235, a controlled and increment volume of liquid hardness titrant solution EDTA from the EDTA container 237, and thoroughly mix these compounds into a sample mixture.

In some embodiments, mixing chamber 240 may include a temperature probe to measure the temperature of the diluted sample solution. Mixing chamber 240 may include a thermal jacket connecting to a recirculating chiller operable to heat or cool the sample mixture to a desired temperature. In some embodiments, mixing chamber 240 includes a pH probe 248 for sensing the pH of the sample mixture, and the pH probe 248 may be coupled to a feedback mechanism for regulating the volume of chemicals dispersed into the mixing chamber 240 from the source of chemicals.

Liquid hardness analyzer 210 includes an automated filter 250 downstream of mixing chamber 240 and having porous filter element through which the diluted sample mixture is passed, after being processed in mixing chamber 240, to obtain the liquid analyte. For example, porous filter element may comprise nylon membrane or other materials with pore size suitable for the mineral sample to be analyzed. For example, the filter pore sizes may be in the range of 0.1 μm to 3.0 μm.

The automated filter 250 is operable to remove coarse particulate and hydrocarbon droplets from the diluted sample mixture and allow the liquid filtrate to pass therethrough. An example of automated filter 250 may be a second ISOLOK™ automated sampler 251 of a style in which as a plunger of the sampler retracts, the front end of plunger collapses and generates pressure. This second ISOLOK™ automated sampler 251 may be coupled to the mixing chamber 240 so that it extracts an aliquot of the diluted sample mixture from the mixing chamber, and that is further coupled to an automated filter changer 252. Once the aliquot of the diluted sample mixture is extracted from the mixing chamber, the plunger of the sampler device retracts that the front end of plunger collapses and generates pressure to propel the aliquot against a filter media to generate particle free filtrate. The system may be configured to automatically replace the filter media with fresh filter media when it has become fouled. For example, the automated filter changer 252 connects to the outlet of the second ISOLOK™ automated sampler 251. When the processor 270 detects that the pressure resistance of filter element has reached a threshold point due to fouling, it may provide instructions to the automated filter changer 252 to switch to a fresh filter element. While the foregoing is an example of an automated filter 250, other embodiments of an automated filter feeding mechanisms may be used that can extract multiple aliquots, filter them into filtrates and convey the filtrates to the spectrophotometer.

Liquid hardness analyzer 210 may include an oil skimming plate 241 installed in the mixing chamber 240 above the automated filter 250 to minimize oil or bitumen droplets enter the automated filter 250. A similar oil skimming plate may be installed above the pH probe 248 in the mixing chamber 240 to minimize oil or bitumen droplets in the sample attach to the pH probe 248.

Liquid hardness analyzer 210 includes spectrophotometer 260 having an optical flowcell that receives the filtrate from the automated filter 250. Spectrophotometer 260 is operable to measure the spectra absorbance of the filtrate in the flowcell at pre-calibrated range of wavelengths, and the spectra absorbance is transmitted to processor 270 for computational analysis. A suitable spectrophotometer 260 for use in the present invention includes but is not limited to a Model CXR-25 Black Comet Spectrophotometer manufactured by Stellar Net USA. A suitable flowcell for use in the present invention includes but is not limited to a Model RK-83057-79 manufactured by Cole-Parmer. The spectra absorbance data is used to determine liquid hardness of the slurry or process water sample, which may be used on its own or in conjunction with other parameters to control water heating equipment such as boilers and the associated process.

Accordingly, the diluted mixture in the mixing chamber 240 is conditioned with chemicals from the chemical containers 233 and 234 and EBT from EBT container 235 until, for example, the diluted mixture reaches a target pH as measured by the pH probe 248. While mixing, EDTA solution is injected in increments into the sample mixture from the EDTA container 237. After each EDTA injection and dispersing, a small aliquot of the sample is withdrawn from the mixing chamber through automated filter 250. The filtrate is transferred by the pressure from automated filter 250 or by a peristatic pump to the spectrophotometer 260 via an optical flowcell where the spectra absorbance of the filtrate is measured.

Processor 270 may be operable to provide instructions to the automated filter 250. Processor 270 may be coupled to the spectrophotometer 260 to receive spectra absorbance measurements and to store such data in memory. Processor 270 may be operable to analyze the stored spectra data to determine a titration endpoint, and to correlate the titration endpoint with the desired parameter to be determined for the sample.

The injection of EDTA solution and the spectra absorbance measurement of each aliquot taken after each such EDTA injection will continue until the measured spectra absorbance indicates that an endpoint has been reached or passed, which indicates total reaction of EDTA with Calcium and Magnesium ions in the liquid and a liquid hardness can be determined, or until enough spectra absorbance data is obtained to be useful in deriving a liquid hardness. The spectra absorbance and the cumulative EDTA volume injected can be correlated to determine the liquid hardness, so to achieve effective process control and water management.

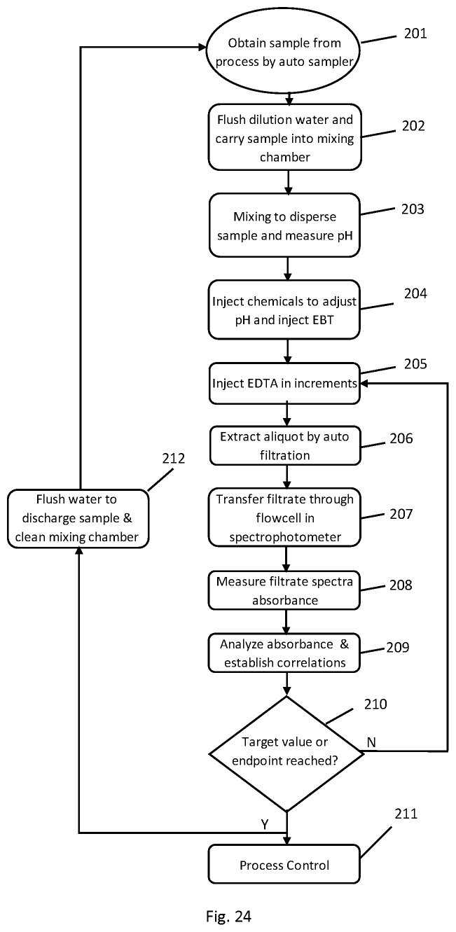

More specifically, with reference to the numbered analysis steps in FIG. 24:
At step 201 the automated sampler 230 takes a controlled volume of mineral slurry or process water sample from a pipeline, container or vessel such as pipeline 220 pursuant to instructions received from the processor 270. (FIG. 15). Processor 270 may be programmed to provide instructions to the automated sampler 230 to obtain a sample at certain times, time intervals, or based on other parameters.
At step 202, upon instructions from the processor 270, a controlled volume of dilution water is injected by the water container 231 into the automated sampler 230 that flushes the sample into the mixing chamber 240 (FIG. 16). The dilution water may be dispensed by a pump controlled by the processor 270.
At step 203 the mixing chamber 240 is equipped with a mixer such as impeller 246 and a pH probe 248. The impeller 246 is activated by the processor 270 to disperse solid particles in the diluted sample mixture and enhance reactions.
At step 204, while mixing, a controlled volume of chemical solutions is injected in increments to the diluted slurry sample until a target pH value is reached as measured by the pH probe. The chemicals injection volume is controlled by the processor 270 which take into consideration the pH measurement. The chemical solutions may be dispensed by a dispenser pump controlled by the processor 270. Also at 204, a controlled volume of EBT solution from the EBT solution container 235 is injected into the chemically conditioned slurry or process water sample. The EBT solution may be dispensed by a dispenser pump controlled by the processor 270 (FIG. 18).
At step 205, while mixing, a controlled volume of EDTA solution is injected in increments from the EDTA solution container 237 to the chemically conditioned slurry or process water sample (FIG. 19). The EDTA solution may be dispensed by a dispenser pump controlled by the processor 270.
At step 206, after each EDTA solution injection, an aliquot of analyte is extracted from the mixing chamber through the automated filter 250.
At step 207, the filtrate is transferred through an optical flowcell of spectrophotometer 260 by the pressure from the automated filter or by a peristaltic pump.
At step 208, the filtrate is measured by the spectrophotometer at pre-calibrated wavelength range and the measured spectra absorbance data is transmitted to the processor 270.
At step 209, the EDTA solution injection continues until either reach a target spectra absorbance value (endpoint) which indicate a completion of the titration, or exceed the endpoint, or enough spectra absorbance data is generated to enable correlation to the liquid hardness and other parameters based on pre-calibration curves.
At step 213, the spectra absorbance value and injected EDTA solution volume, along with slurry or process water properties measured by other instruments installed on the system, are used to correlate and determine the liquid sample's liquid hardness and other values.
At step 214, the liquid hardness and other values are used as input variables for feedback or feed forward systems for controlling the process parameters, such as but not limited to, water softening chemical dosages, slurry or process water or water softening chemical mass or volumetric flowrates, etc.
At step 215, after the liquid pH and hardness values are determined, a controlled volume of flushing (dilution) water is injected into the mixing chamber through the automated sampler to remove the spent slurry or process water sample via a drainage port at the bottom of mixing chamber (FIG. 20).
At step 216, the flushing water also cleans the automated sampler, the mixer impeller and pH probe, the automated filter, and the mixing chamber interior by engaging the mixer; the analyzer is ready to analyze the next sample (FIG. 20).

According to ASTM D1126-17, the hardness of water can be determined using the following equation:

Hardness,epm=20 C/S 15

Where
epm=equivalent parts per million; milliequivalents per litre
C=standard Na₂H₂EDTA solution added in titrating hardness, mL, and
S=sample volume, mL

Other types of water hardnesses, such as Calcium hardness, Magnesium hardness and hardness as Calcium Carbonate use the similar calculation as the hardness of water, the key is to obtain the volume of standard Na₂H₂EDTA (a.k.a. EDTA) added into the liquid sample when reaching the titration endpoint; or in other words when Calcium and Magnesium ions are fully un-complexed with the EBT by the addition of EDTA.

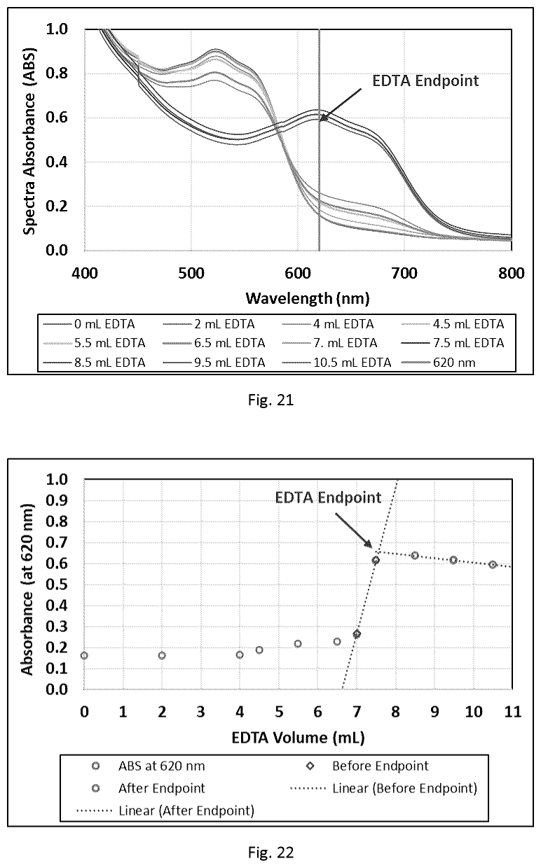

In the online mineral slurry and process water hardness analyzer of the present invention, the endpoint is determined by correlating the spectra absorbance vs. EDTA volume titrated into the sample, as shown in FIGS. 21 and 22, which enable the process to be automated and on-line.

### Example 2

An oil sands SAGD process water is tested using the automated and on-line liquid hardness analyzer as described herein. FIG. 21 shows a group of spectra absorbance curves, the spectra absorbance (ABS) at 620 nm increases with the increasing EDTA volume titrated until reaching an endpoint. FIG. 22 shows the endpoint can be determined by plotting the ABS vs. the cumulative EDTA volume titrated, the joint between two distinctive curves is the titration endpoint (at 7.5 mL EDTA) where ABS increased drastically with only small increase of EDTA volume. Therefore, the endpoint can be determined by correlating the ABS vs. cumulative EDTA volume, which is then used to calculate the hardness of the process water using the above Equation 15. The liquid hardness of slurry or process water is determined automatically and the analyzed can be installed and operated on-line to an active process

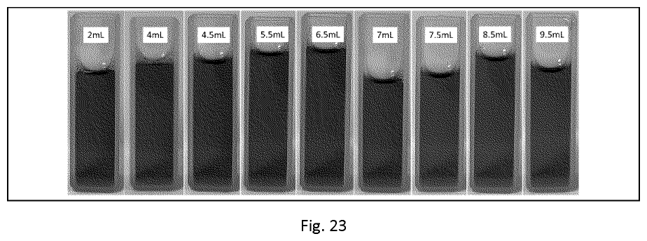

Liquid hardness analyzer 210 provides a new liquid hardness method and apparatus that utilizes a spectrophotometer to determine the titration endpoint as a replacement of the conventional titration procedures outlined by ASTM D1126-17. This is achieved by a series of online and automated procedures, including automated withdraw of sample from a process, condition it in a mixing chamber, extract a filtrate by an automated filter and analyze it by a spectrometer, then correlate the filtrate spectra absorbance with cumulative EDTA volume injected until reach the titration endpoint. The analyzer can be used automatically and continuously, and it improves the accuracy by eliminating human subjective and visual endpoint detection as shown in FIG. 23. Furthermore, the spectra absorbance data, even before reaching the endpoint, can be used for correlation and process control, which can replace or supplement the endpoint detection and shorten the measuring time.

The present invention also provides close to real-time measurement of total hardness, Calcium and Magnesium hardness, the liquid hardness as Calcium Carbonate, Calcium and Magnesium ion concentration etc., this enables automated and online monitoring of process pH and hardness in liquid of slurry or process water.

Some general implementations of the present invention may be as follows:

Applications where an online measurement of hardness in water is required to monitor the quality of water supply to heating equipment such as boiler and heat exchanger. The current ICP method requires delicate instrumentations and well trained personnel to operate, the requirement for sample is very high that it need considerable time to process and to prepare the sample, such that it cannot be converted into an automated and online method; the ICP is also not suitable to be operated near or at the process sites as it is not robust enough to be developed as an online method as it cannot be accommodated to harsh process conditions such as high temperature, high pressure and dusty environment.

While embodiments of the invention have been described and illustrated, such embodiments should be considered illustrative of the invention only. The invention may include variants not described or illustrated herein in detail. Thus, the embodiments described and illustrated herein should not be considered to limit the invention.

What is claimed is:

1. An automated pH analyzer for determining the pH in a mineral slurry or process water in a vessel or passing through a conduit, the apparatus comprising:
a processor operable to manage the operations associated with the apparatus;
an automated sampler coupled to the vessel or conduit and operable to extract a sample of a determined volume of the slurry or process water from the vessel or conduit, the automated sampler being under control of the processor;
a water source under control of the processor and operable to deliver a known volume of water of a known pH into the sample;
a mixing chamber that receives the known volume of water and the sample;
an agitator operable to agitate the sample and the known volume of water in the mixing chamber to produce a diluted sample mixture;
an automated filter operable to extract an aliquot of the diluted sample mixture from the mixing chamber and to filter the aliquot to produce a filtrate;
a pH probe after the automated filter to measure the pH of filtrate; and
a pH probe within the mixing chamber operable to measure a pH of the diluted sample mixture, wherein the measurement is used in any one or more of following: to calculate the pH of the extracted sample, and to alter in near real time a process control of the a mineral processing operation related to the mineral slurry or process water.

2. The apparatus as claimed in claim 1, wherein the apparatus is online such that the sample is withdrawn from an online active process.

3. The apparatus as claimed in any one of claims 1-2, wherein the processor is operable to instruct the automated sampler to extract the sample from the vessel or conduit.

4. The apparatus as claimed in any one of claims 1-3, wherein the water source delivers the known volume and known pH of water to the automated sampler after the sample has been extracted to flush the sample out of the automated sampler and into the mixing chamber.

5. The apparatus as claimed in any one of claims 1-4, wherein the processor is operable to instruct the water source to deliver the known volume of water to the automated sampler.

6. The apparatus as claimed in any one of claims 1-5, wherein the water source cooperates with the automated sampler to deliver the volume of water into the extracted sample to flush it out of the automated sampler to clean the automated sampler thereby ready it for obtaining a subsequent sample of slurry or process water.

7. The apparatus as claimed in any one of claims 1-6, wherein the agitator is controlled by the processor.

8. The apparatus as claimed in claim 7, wherein the processor is operable to activate the agitator to mix the sample mixture after the sample mixture is received in the mixing chamber.

9. The apparatus as claimed in any one of claims 1-8, wherein the processor is operable to receive the pH measurement of the diluted sample mixture from the pH probe within the mixing chamber after a period of agitation of the diluted sample mixture.

10. The apparatus as claimed in any one of claims 1-9, wherein the water source is operable to flush water through one or both of the automated sampler and the mixing chamber, after the pH measurement of the diluted sample mixture, to clean one or both of the automated sampler and the mixing chamber in preparation for processing a subsequent sample.

11. The apparatus as claimed in claim 10, wherein the processor is operable to activate the agitator while the water source is operable to flush water through the mixing chamber.

12. The apparatus as claimed in any one of claims 1-11, wherein the automated filter comprises: a second automated sampler coupled to the mixing chamber and operable to extract the aliquot from the mixing chamber after mixing the process sample with dilution water; and a filter element downstream of the automated filter, wherein to produce a filtrate and the pH of filtrate is measured by the pH probe installed after the automated filter.

13. The apparatus as claimed in any one of claims 1-12, wherein the processor is operable to calculate the pH of the sample using the known volume of the sample, the known volume of the water delivered into the sample, the known pH of the volume of water delivered into the sample, the measured pH of the diluted sample mixture, and the measured pH of the filtrate.

14. A method of determining a pH in a mineral slurry or process water in a vessel or passing through a conduit, the method comprising:
a. coupling an automated sampler with the vessel or conduit such that the automated sampler is operable to extract a sample of a known volume of the slurry or process water from the vessel or conduit;
b. providing instructions from a processor to the automated sampler to extract the sample;
c. flushing the sample from the automated sampler into a mixing chamber with a known volume of water having a known pH from a water source under control of the processor;
d. mixing the sample and the volume of water with an agitator in the mixing chamber under control of the processor to produce a diluted sample mixture;
e. measuring a pH of the diluted sample mixture with a pH probe in the mixing chamber under control of the processor;
f. extract an aliquot of the sample mixture, filter through an automated filter and measure the pH of filtrate by a pH probe after the automated filter; and
g. analyzing the pH measurement of the diluted sample mixture and filtrate with the processor to determine a pH of the extracted process sample.

15. The method of claim 14, further comprising flushing water from the water source under control of the processor through the automated sampler and mixing chamber after step (f) to expel remnants of the diluted sample mixture therefrom in preparation for processing a subsequent sample.

16. An automated quantitative volumetric titration analyzer for performing automated quantitative volumetric titrations of a mineral slurry or process water in a vessel or passing through a conduit, the apparatus comprising:
a processor operable to manage the operations associated with the apparatus;
an automated sampler coupled to the vessel or conduit and operable to extract a sample of a determined volume of the slurry or process water from the vessel or conduit, the automated sampler being under control of the processor;
a water source under control of the processor and operable to deliver a known volume of water into the sample;
a titrant solution source under control of the processor and operable to deliver a known volume of titrant solution to the sample;
a mixing chamber that receives the sample, the water, and the titrant solution;
an agitator operable to agitate the sample, the water, and the titrant solution in the mixing chamber to produce a diluted sample mixture;
an automated filter operable to extract an aliquot of the diluted sample mixture from the mixing chamber and to filter the aliquot to produce a filtrate; and
a spectrophotometer having an optical flowcell that receives the filtrate from the automated filter and operable to measure a spectra absorbance of the filtrate in the optical flowcell using at least one wavelength to obtain spectra absorbance data of the filtrate.

17. The apparatus as claimed in claim 16, wherein the apparatus is online such that the sample is withdrawn from an online active process.

18. The apparatus as claimed in any one of claims 16-17, further comprising a source of chemicals under control of the processor and operable to deliver chemicals into the mixing chamber for chemically conditioning the sample mixture.

19. The apparatus as claimed in claim 18, further comprising a pH probe within the mixing chamber operable to measure a pH of the diluted sample mixture, wherein the processor is operable to control the delivery of chemicals to the sample mixture based on the pH measurement.

20. The apparatus as claimed in any one of claims 16-19, further comprising: a recirculating chiller coupled to the mixing chamber operable to heat or cool the sample mixture; a temperature probe in the mixing chamber operable to measure a temperature of the sample mixture; and wherein the processor is operable to receive the temperature measurement from the temperature probe and to activate the recirculating chiller based on the temperature measurement to achieve a desired temperature of the sample mixture.

21. The apparatus as claimed in any one of claims 16-20, wherein the processor is operable to instruct the automated sampler to extract the sample from the vessel or conduit.

22. The apparatus as claimed in any one of claims 16-21, wherein the water source delivers the known volume of water to the automated sampler after the sample has been extracted to flush the sample out of the automated sampler and into the mixing chamber.

23. The apparatus as claimed in any one of claims 16-22, wherein the processor is operable to instruct the water source to deliver the known volume of water to the automated sampler.

24. The apparatus as claimed in any one of claims 16-23, wherein the water source cooperates with the automated sampler to deliver the volume of water into the extracted sample to flush it out of the automated sampler to clean the automated sampler thereby ready it for obtaining a subsequent sample of slurry or process water.

25. The apparatus as claimed in any one of claims 16-24, wherein the agitator is controlled by the processor.

26. The apparatus as claimed in claim 25, wherein the processor is operable to activate the agitator to mix the sample mixture after the sample mixture is received in the mixing chamber.

27. The apparatus as claimed in any one of claims 16-26, wherein the water source is operable under control of the processor to flush water through one or both of the automated sampler and the mixing chamber to clean one or both of the automated sampler and the mixing chamber in preparation for processing a subsequent sample.

28. The apparatus as claimed in claim 27, wherein the processor is operable to activate the agitator while the water source is operable to flush water through the mixing chamber.

29. The apparatus as claimed in any one of claims 16-28, wherein the automated filter comprises: a second automated sampler coupled to the mixing chamber and operable to extract the aliquot from the mixing chamber after each delivery of the titrant solution;
and a filter element downstream of the second automated sampler, wherein the second automated sampler pumps the aliquot through the filter element and the filtrate to the optical flowcell for obtaining spectra absorbance measurements of each filtrate.

30. The apparatus as claimed in claim 29, wherein the automated filter includes a pressure sensor that senses pressure of the aliquot upstream of the filter element; and a mechanism operable to replace the filter element with a fresh filter element as a result of a signal from the pressure sensor that the pressure of the aliquot has increased beyond a threshold pressure.

31. The apparatus as claimed in any one of claims 16-30, wherein the processor is operable to determine a titration endpoint from the spectra absorbance data.

32. The apparatus as claimed in any one of claims 16-31, wherein the processor is operable to control a processing of the mineral slurry or process water or to control in near real time a processing operation related to the mineral slurry or process water, based on the spectra absorbance data.

33. The apparatus as claimed in claim 32, wherein if the processor determines the titration endpoint has not been reached, the processor is further operable: to instruct the titrant solution source to deliver an additional known volume of titrant solution to the dilute sample mixture; thereafter to instruct the automated filter to obtain a subsequent aliquot of the diluted sample mixture and filter same to produce a subsequent filtrate; and thereafter to instruct the spectrophotometer to measure a spectra absorbance of the subsequent filtrate to obtain a subsequent spectra absorbance data; and thereafter determine if the titration endpoint has been reached from the subsequent spectra absorbance data.

34. The apparatus as claimed in claim 32, wherein if the processor determines the titration endpoint has been reached and/or enough titration data has been obtained, the processor is further operable to instruct the water source to flush water through one or both of the automated sampler and the mixing chamber to clean one or both of the automated sampler and the mixing chamber in preparation for processing a subsequent sample of mineral slurry or process water.

35. A method of automatically performing a quantitative volumetric titration on a mineral slurry or process water in a vessel or passing through a conduit, the method comprising the steps of:
a. coupling an automated sampler with the vessel or conduit such that the automated sampler is operable to extract a sample of a known volume of the slurry or process water from the vessel or conduit;
b. providing instructions from the processor to the automated sampler to extract the sample;
c. flushing the sample from the automated sampler into a mixing chamber with a known volume of water from a water source under control of the processor;
d. mixing the sample and water in the mixing chamber to produce a diluted sample mixture;
e. adding a known volume of chemical and indicator solutions into the diluted sample mixture from chemical and indicator solution sources under control of the processor;
f. adding a known volume of a titrant solution into the diluted sample mixture from a titrant solution source under control of the processor;
g. filtering an aliquot of the diluted sample mixture through filter media of an automated filter and directing a filtrate of the aliquot into an optical flowcell of a spectrophotometer;
h. measuring spectra absorbance of the filtrate under control of the processor to obtain spectra absorbance data of the filtrate, and storing the spectra absorbance data in memory;
i. repeating steps (f) to (h) until a target spectra absorbance value or a plurality of target spectra absorbance values is reached to obtain a spectra absorbance data set;
j. flushing water through the automated sampler and mixing chamber to expel remnants of the slurry sample and process solutions therefrom in preparation for processing a subsequent sample; and
k. analyzing the spectra absorbance data set and using a result of the analysis in controlling processing of the mineral slurry or process water or controlling other aspects of a mineral processing operation related to the mineral slurry or process water.

36. The method as claimed in claim 35, further comprising a step of homogenizing the sample mixture before and after adding titrant solution to disperse particles in the sample mixture.

37. The method of claim 36, wherein the step of homogenizing the sample mixture takes place in the mixing chamber.

38. The method as claimed in any one of claims 35-37, further comprising a step of measuring a density of the slurry sample in the vessel or conduit near the analyzer.

39. The method as claimed in any one of claims 35-38, further comprising regulating a temperature of the sample mixture in the mixing chamber under control from the processor.

40. The method as claimed in claim 39 wherein the step of regulating a temperature of the diluted sample mixture comprises establishing a flow of hot fluid or cold fluid through a fluid jacket provided around at least a portion of the mixing chamber.

41. The method as claimed in any one of claims 35-40 further comprising repeating steps (b) to (j) to obtain a data set on a desired number of samples.

42. An automated liquid hardness analyzer for determining the hardness in a mineral slurry or process water in a vessel or passing through a conduit, the apparatus comprising:
a processor operable to manage the operations associated with the apparatus;
an automated sampler coupled to the vessel or conduit and operable to extract a sample of a determined volume of the slurry or process water from the vessel or conduit, the automated sampler being under control of the processor;
a water source under control of the processor and operable to deliver a known volume of water into the sample;
an Eriochrome Black T (EBT) solution source under control of the processor and operable to deliver a known volume of EBT solution to the sample;
an Ethylenediamine Tetraacetic Acid (EDTA) solution source under control of the processor and operable to deliver a known volume of EDTA solution to the sample;
a mixing chamber that receives the sample, the water, the EBT solution and the EDTA solution;
an agitator operable to agitate the sample, the water, the EBT solution and the EDTA solution in the mixing chamber to produce a diluted sample mixture;
an automated filter operable to extract an aliquot of the diluted sample mixture from the mixing chamber and to filter the aliquot to produce a filtrate;
a spectrophotometer having an optical flowcell that receives the filtrate from the automated filter and operable to measure a spectra absorbance of the filtrate in the optical flowcell using at least one wavelength to obtain spectra absorbance data of the filtrate; and
wherein the processor if operable to determine the EDTA titration endpoint from the spectra absorbance data and to correlate the EDTA titration endpoint and the cumulative EDTA solution volume to a liquid hardness value of the extracted sample.

43. The apparatus as claimed in claim 42, wherein the apparatus is online such that the sample is withdrawn from an online active process.

44. The apparatus as claimed in any one of claims 42-43, further comprising a source of chemicals under control of the processor and operable to deliver chemicals into the mixing chamber for chemically conditioning the sample mixture.

45. The apparatus as claimed in claim 44, further comprising a pH probe within the mixing chamber operable to measure a pH of the diluted sample mixture, wherein the processor is operable to control the delivery of chemicals to the sample mixture based on the pH measurement.

46. The apparatus as claimed in any one of claims further comprising: a recirculating chiller coupled to the mixing chamber operable to heat or cool the sample mixture; a temperature probe in the mixing chamber operable to measure a temperature of the sample mixture; and wherein the processor is operable to receive the temperature measurement from the temperature probe and to activate the recirculating chiller based on the temperature measurement to achieve a desired temperature of the sample mixture.

47. The apparatus as claimed in any one of claims 42-46, wherein the processor is operable to instruct the automated sampler to extract the sample from the vessel or conduit.

48. The apparatus as claimed in any one of claims 42-47, wherein the water source delivers the known volume of water to the automated sampler after the sample has been extracted to flush the sample out of the automated sampler and into the mixing chamber.

49. The apparatus as claimed in any one of claims 42-48, wherein the processor is operable to instruct the water source to deliver the known volume of water to the automated sampler.

50. The apparatus as claimed in any one of claims 42-49, wherein the water source cooperates with the automated sampler to deliver the volume of water into the extracted sample to flush it out of the automated sampler to clean the automated sampler thereby ready it for obtaining a subsequent sample of slurry or process water.

51. The apparatus as claimed in any one of claims 42-50, wherein the agitator is controlled by the processor.

52. The apparatus as claimed in claim 51, wherein the processor is operable to activate the agitator to mix the sample mixture after the sample mixture is received in the mixing chamber.

53. The apparatus as claimed in any one of claims 42-52, wherein the water source is operable under control of the processor to flush water through one or both of the automated sampler and the mixing chamber to clean one or both of the automated sampler and the mixing chamber in preparation for processing a subsequent sample.

54. The apparatus as claimed in claim 53, wherein the processor is operable to activate the agitator while the water source is operable to flush water through the mixing chamber.

55. The apparatus as claimed in any one of claims 42-54, wherein the automated filter comprises: a second automated sampler coupled to the mixing chamber and operable to extract the aliquot from the mixing chamber after each delivery of the EDTA solution; and a filter element downstream of the second automated sampler, wherein the second automated sampler pumps the aliquot through the filter element and the filtrate to the optical flowcell for obtaining spectra absorbance measurements of each filtrate.

56. The apparatus as claimed in claim 55, wherein the automated filter includes a pressure sensor that senses pressure of the aliquot upstream of the filter element; and a mechanism operable to replace the filter element with a fresh filter element as a result of a signal from the pressure sensor that the pressure of the aliquot has increased beyond a threshold pressure.

57. The apparatus as claimed in any one of claims 42-56, wherein the processor is operable to control a processing of the mineral slurry or process water in near real time based on the determined hardness value.

58. The apparatus as claimed in any one of claims 42-57, wherein if the processor determines the EDTA titration endpoint has not been reached, the processor is further operable: to instruct the EDTA solution source to deliver an additional known volume of EDTA solution to the dilute sample mixture; thereafter to instruct the automated filter to obtain a subsequent aliquot of the diluted sample mixture and filter same to produce a subsequent filtrate; and thereafter to instruct the spectrophotometer to measure a spectra absorbance of the subsequent filtrate to obtain a subsequent spectra absorbance data; and thereafter determine if the titration endpoint has been reached from the subsequent spectra absorbance data.

59. The apparatus as claimed in any one of claims 42-58, wherein if the processor determines the EDTA titration endpoint has been reached, the processor is further operable to instruct the water source to flush water through one or both of the automated sampler and the mixing chamber to clean one or both of the automated sampler and the mixing chamber in preparation for processing a subsequent sample of mineral slurry or process water.

60. A method of automatically determining a liquid hardness value of a mineral slurry or process water in a vessel or passing through a conduit, the method comprising the steps of:
a. coupling an automated sampler with the vessel or conduit such that the automated sampler is operable to extract a sample of a known volume of the slurry or process water from the vessel or conduit;
b. providing instructions from the processor to the automated sampler to extract the sample;
c. flushing the sample from the automated sampler into a mixing chamber with a known volume of water from a water source under control of the processor;
d. mixing the sample and water in the mixing chamber to produce a diluted sample mixture;
e. adding known volume of chemical solutions into the diluted sample mixture from chemical solution source under control of the processor;
f. adding a known volume of Eriochrome Black T (EBT) solution into the diluted sample mixture from an EBT solution source under control of the processor;
g. adding a known volume of Ethylenediamine Tetraacetic Acid (EDTA) solution into the diluted sample mixture from an EDTA solution source under control of the processor;
h. filtering an aliquot of the diluted sample mixture through filter media of an automated filter and directing a filtrate of the aliquot into an optical flowcell of a spectrophotometer;
i. measuring spectra absorbance of the filtrate under control of the processor to obtain spectra absorbance data of the filtrate, and storing the spectra absorbance data in memory;
j. repeating steps (g) to (i) until a target spectra absorbance value or a plurality of target spectra absorbance values is reached to obtain a spectra absorbance data set;
k. flushing water through the automated sampler and mixing chamber to expel remnants of the sample and process solutions therefrom in preparation for processing a subsequent sample; and
l. analyzing the spectra absorbance data set and using a result of the analysis in determining a liquid hardness value for the extracted sample.

61. The method as claimed in claim 60, further comprising a step of homogenizing the sample mixture before and after adding titrant solution to disperse particles in the sample mixture.

62. The method of claim 60, wherein the step of homogenizing the sample mixture takes place in the mixing chamber.

63. The method as claimed in any one of claims 60-62, further comprising a step of measuring a density of the slurry sample in the vessel or conduit near the analyzer.

64. The method as claimed in any one of claims 60-63, further comprising regulating a temperature of the sample mixture in the mixing chamber under control from the processor.

65. The method as claimed in claim 64 wherein the step of regulating a temperature of the diluted sample mixture comprises establishing a flow of hot fluid or cold fluid through a fluid jacket provided around at least a portion of the mixing chamber.

66. The method as claimed in any one of claims 60-65 further comprising repeating steps (b) to (j) to obtain a data set on a desired number of samples.
